(19) United States
(12) Patent Application Publication
(10) Pub. No.: US 2009/0123919 A1
(43) Pub. Date: May 14, 2009

(54) Diagnosing Pathological Conditions Using Interallelic Epigenetic Variations

(75) Inventor: Lydia Avivi, Carmei Yosef (IL)

Correspondence Address: MARTIN D. MOYNIHAN d/b/a PRTSI, INC., P.O. BOX 16446, ARLINGTON, VA, 22215, US

(73) Assignee: Allelis Diagnostics Ltd., Karmei Yoseef (IL)

(21) Appl. No.: 11/922,510

(22) PCT Filed: Jun. 21, 2006
(86) PCT No.: PCT/IL2006/000725

§ 371 Date: Dec. 19, 2007

Related U.S. Application Data
(60) Provisional application No. 60/692,255, filed on Jun. 21, 2005.
(60) Provisional application No. 60/702,635, filed on Jul. 27, 2005.

(51) Int. Cl. C12Q 1/68

(52) U.S. Cl. 435/6

(57) ABSTRACT

The present invention is of interallelic epigenetic alterations in cells of cancer-stricken individuals which can be used for diagnosing cancer or cancer risk. In addition, the present invention is of interallelic epigenetic pattern alterations and/or interallelic replication pattern alterations in cells of a conceptus having imbalanced chromosome(s) or in maternal cells of the pregnant female carrying the conceptus, which can be used for prenatal diagnosis of chromosomal imbalances in the conceptus. Moreover, the present invention is of interallelic epigenetic pattern alterations and/or interallelic replication pattern alterations in cells of individuals having a chromosomal imbalance mosaicism which can be used for diagnosing chromosomal imbalance mosaicism, either prenatally or after birth.

### FIELD AND BACKGROUND OF THE INVENTION

The present invention relates to methods of diagnosing pathological conditions associated with interallelic epigenetic changes such as cancer and fetal chromosomal imbalances and more particularly, to the use of DNA methylation and/or chromatin profile assays for the detection of cancer and/or chromosomal imbalances in a fetus.

Cancer Diagnosis

The diagnosis of cancer, and especially of solid tumors, relies on direct sampling of tumor cells using invasive procedures such as core needle biopsy. Efforts to identify abnormalities in unaffected, easily attained tissues such as peripheral blood of patients with solid tumors have been disappointing so far. For example, the level of prostate-specific antigen (PSA) in blood, which is largely used for the detection of prostate cancer, provides a positive predictive value in only about 20-30% of the cases. Thus, there is a need to develop new diagnostic approaches for the detection of cancer.

Epigenetic changes, resulting from DNA and histone modifications, may lead to heritable silencing of genes without a change in their coding sequence (Egger et al., 2004). These changes are usually established in parental germ cells and inherited post fertilization to the offspring during successive cell divisions.

The most prominent DNA epigenetic modification is by methylation typically on CpG islands. These are sequence regions of more than 500 base pairs in size with a GC content greater than 55%, normally kept free of DNA methylation and are the sites of DNA methylation in various conditions or pathologies. CpG islands are located within the promoter regions of about 40% of mammalian genes and when methylated, cause stable transcriptional silencing through successive generations of somatic divisions.

Epigenetic changes may also occur on chromatin. Chromatin modifications such as histone acetylation, deacetylation, methylation or demethylation of conserved lysine residues on the amino-terminal tail domains are associated with transcriptional activation or silencing.

Epigenetic changes leading to silencing of genes such as tumor suppressors or oncogenes can have a major role in the development of human cancer (Baylin and Herman, 2000; Jones and Baylin, 2002; Kane et al., 1997). Indeed, inhibitors of DNA methylation were shown capable of reactivating the expression of genes undergoing epigenetic silencing (e.g., p16), particularly if the silencing has occurred in a pathological situation (Baylin S, et al., 2000).

The present inventors have previously disclosed a simple and non-invasive method of detecting cancer and cancer risk based on the level of asynchronous replication of alleles in blood samples (PCT Publication No. WO02/023187 A2 and U.S. Pat. No. 0,680,3195 to the present inventor). However, although the inventors called for determination of the coordination between alleles of one or more DNA loci, wherein the coordination is methylation, hypermethylation, hypomethylation, gene expression or fidelity of chromosome segregation, they teach diagnosing cancer by detecting the replication pattern (using the FISH replication assay) of various loci of cells in cancer stricken individuals.

Thus, to date, a method diagnosing cancer by analyzing interallelic epigenetic modifications occurring in cells of cancer stricken individuals was not taught.

Prenatal Diagnosis

Approximately 3% of viable fetuses are born with a severe anomaly. Moreover, the risk of giving birth to an infant with a chromosomal defect resulting from a division error or chromosomal disjunction failure increases with age.

A variety of invasive and non-invasive techniques are currently known for prenatal diagnosis. The invasive techniques include amniocentesis, chorionic villus sampling (CVS), cordocenthesis (sampling of fetal cord blood) and foetoscopy (the introduction of a tube with optic fibers through the uterus allowing biopsy or surgical interventions). The non-invasive techniques include ultrasonography, analysis of circulating fetal blood cells in maternal blood, and detection of biochemical products in maternal serum (e.g., the quadruple test).

Genetic testing using cytogenetic (e.g., karyotype and FISH) and/or DNA (e.g., single-gene disorders) analyses is performed on fetal cells obtained by amniocentesis or CVS, and in rare cases also by cordocenthesis. However, while karyotype analysis enables the identification of gross chromosomal alterations (e.g., loss or gain of whole or significant portions of a chromosome), FISH analysis or DNA analysis performed using locus- or gene-specific probes are selected based on prior knowledge of chromosomal aberrations and/or DNA mutations present in affected siblings.

The analysis of fetal DNA present in the maternal plasma represents a major advantage over conventional invasive methods of prenatal diagnosis. However, due to the coexisting maternal DNA, the accuracy of such analysis is limited. In addition, detection of fetal abnormalities using rare fetal blood cells present in the maternal blood was also demonstrated, especially using FISH analysis. However, since fetal cells in the maternal circulation are rare, their laborious isolation limits their use in prenatal diagnosis.

Thus, while invasive techniques are relatively accurate but are associated with increased risk of fetal mortality, the currently available non-invasive tests are limited by low sensitivity and the difficulties associated with isolation of rare cells. In addition, although cytogenetic and DNA analyses enable the identification of a wide variety of genetic syndromes or disorders, a significant portion of the pregnancies which involved prenatal diagnosis results in the birth of abnormal infants having genetic disorders caused by microdeletions, translocations or small duplications which were misdiagnosed.

Evidence that chromosomally imbalance genomes may display alterations in the temporal order of allelic replication of genes not associated with the aberrant chromosome(s) was obtained using the FISH replication assay (Amiel et al. 1998a, 1999; Goldshtein 2004). When applied to amniotic fluid cells it was shown that in various trisomies including Down syndrome (trisomy 21), Edwards' syndrome (trisomy 18) and Patau's syndrome (trisomy 13), the replication-timing properties of several autosomal biallelically-expressed genes, which are not located on the triplicated chromosome(s) is altered. In addition, monoallelically-expressed genes (e.g., SNRPN and XIST), not associated with the triplicated chromosome of Down syndrome, exhibited loss of their replication timing properties (i.e., from an asynchronous pattern to a synchronous pattern) (Senn 2003). Moreover, in both lymphocytes and amniocytes of subjects with Turner syndrome (X-monosomy) modifications in the replication timing of biallelically-expressed genes (such as RB1, TP53 and CMYC) were demonstrated (Reish et al 2002; Goldshtein 2004). Furthermore, even genomes carrying micro deletions and/or small duplications show alterations in allelic replication of genes not obviously associated with the aberrant part of the chromosome (Ofir et al. 1999; Amiel at al. 2001, 2002).

Interestingly, an aberrant replication phenotype was observed in normal (euploid) amniocytes cultured in the presence of aneuploid amniocytes, derived from either Down, Edwards, Patau or Turner syndrome fetuses (Goldshtein, 2004). In addition, Senn et al., 2003, demonstrated that the loss of the inherent temporal order of allelic replication in the aneuploid genotypes can be reversed in the presence of 5-azacytidine, an inhibitor of DNA methylation.

However, although these studies demonstrated alterations in replication pattern of various loci in individuals with chromosomal aberrations, to date, no method of prenatally diagnosing a fetus by analyzing the interallelic epigenetic modifications and/or interallelic replication pattern in cells of the fetus and/or in maternal cells of the pregnant female carrying the fetus was ever taught or suggested.

Altogether, although alterations in replication synchrony of various genes were documented in both cancer cells and individuals with chromosomal aberrations, a method of cancer diagnosis and/or prenatal diagnosis of a fetus which is exclusively based on analyzing the interallelic epigenetic pattern of various loci, which are not directly associated with disease onset or progression in cells of individuals affected with cancer and/or of a fetus or the pregnant female carrying the fetuses with chromosomal imbalances was not taught.

There is thus a widely recognized need for, and it would be highly advantageous to have, a non-invasive and highly reliable method of cancer diagnosis and/or prenatal diagnosis devoid of the above limitations.

### SUMMARY OF THE INVENTION

According to one aspect of the present invention there is provided a method of diagnosing cancer, the method comprising, determining in at least one locus of a cell of an individual in need thereof an interallelic epigenetic pattern, wherein an alteration in the interallelic epigenetic pattern compared to the interallelic epigenetic pattern of the at least one locus in a cell of an unaffected individual is indicative of the cancer, thereby diagnosing the cancer.

According to another aspect of the present invention there is provided a method of prenatally identifying a chromosomal imbalance in a conceptus, the method comprising, determining in at least one locus of a maternal cell of a pregnant female carrying the conceptus an interallelic epigenetic pattern, wherein an alteration in the interallelic epigenetic pattern compared to the interallelic epigenetic pattern of the at least one locus in a cell of an individual devoid of the chromosomal imbalance is indicative the chromosomal imbalance in the conceptus.

According to yet another aspect of the present invention there is provided a method of prenatally identifying a chromosomal imbalance in a conceptus with the proviso that the chromosomal imbalance is not an imprinting-associated chromosomal imbalance, the method comprising, determining in at least one locus of a cell of the conceptus or a maternal cell of a pregnant female carrying the conceptus an interallelic epigenetic pattern, wherein an alteration in the interallelic epigenetic pattern compared to the interallelic epigenetic pattern of the at least one locus in a cell of an individual devoid of the chromosomal imbalance is indicative of the chromosomal imbalance in the conceptus.

According to still another aspect of the present invention there is provided a method of prenatally identifying an imprinting-associated chromosomal imbalance in a conceptus, the method comprising determining in at least one locus of a cell of the conceptus or a maternal cell of a pregnant female carrying the conceptus an interallelic epigenetic pattern with the proviso that the at least one locus is a monoallelically expressed locus, wherein an alteration in the interallelic epigenetic pattern compared to the interallelic epigenetic pattern of the at least one locus in a cell of an individual devoid of the chromosomal imbalance is indicative of the imprinting-associated chromosomal imbalance in the conceptus.

According to an additional aspect of the present invention there is provided a method of identifying a chromosomal imbalance mosaicism in an individual in need thereof, the method comprising determining in at least one locus of a cell of the individual an interallelic epigenetic pattern, wherein an alteration in the interallelic epigenetic pattern compared to the interallelic epigenetic pattern of the at least one locus in a cell of an individual devoid of the chromosomal imbalance mosaicism is indicative the chromosomal imbalance mosaicism in the individual.

According to yet an additional aspect of the present invention there is provided a method of prenatally identifying a chromosomal imbalance of a conceptus with the proviso that the chromosomal imbalance is not an imprinting-associated chromosomal imbalance, the method comprising, determining in at least one locus of a cell of the conceptus an interallelic replication pattern, wherein an alteration in the interallelic replication pattern compared to the interallelic replication pattern of the at least one locus in a cell of an individual devoid of the chromosomal imbalance is indicative of the chromosomal imbalance in the conceptus.

According to still an additional aspect of the present invention there is provided a method of prenatally identifying a chromosomal imbalance of a conceptus, the method comprising, determining in at least one locus of a maternal cell of a pregnant female carrying the conceptus an interallelic replication pattern, wherein an alteration in the interallelic replication pattern compared to the interallelic replication pattern of the at least one locus in a cell of an individual devoid of the chromosomal imbalance is indicative of the chromosomal imbalance in the conceptus.

According to further features in preferred embodiments of the invention described below, the epigenetic pattern is selected from the group consisting of DNA methylation, chromatic methylation and chromatin acetylation.

According to still further features in the described preferred embodiments the cell comprises a cell culture.

According to still further features in the described preferred embodiments the cell culture comprises a mitotic division stimulating agent.

According to still further features in the described preferred embodiments the cell culture comprises cells following at least one population doubling.

According to still further features in the described preferred embodiments the cell culture further comprises an epigenetic modifier agent.

According to still further features in the described preferred embodiments the epigenetic modifier agent is a methylation modifying agent and/or an acetylation modifying agent.

According to still further features in the described preferred embodiments the methylation modifying agent is a DNA methylation inhibitor, histone methylation inhibitor and/or histone demethylation inhibitor.

According to still further features in the described preferred embodiments the DNA methylation inhibitor is selected from the group consisting of 5-azacytidine (5-aza-CR), 5-aza-2′deoxycytidine (5-aza-CdR), 5 fluorocytosine, pseudoisocytosine, Zebularine, Procainamide, polyphenol (−)-epigallocatechin-3-gallate (EGCG), and Psammaplin.

According to still further features in the described preferred embodiments the acetylation modifying agent is a histone deacetylase (HDAC) inhibitor, a histone acetyltransferase (HAT) inhibitor, histone deacetylase and histone acetyltransferase.

According to still further features in the described preferred embodiments the histone deacetylase (HDAC) inhibitor is Trichostatin A (TSA), Sodium butyrate, suberoylanilide hydroxamic acid (SAHA) and N-nitroso-n-methylurea.

According to still further features in the described preferred embodiments the histone acetyltransferase (HAT) inhibitor is Polyisoprenylated Benzophenone (Garcinol) and Set/TAF-1 beta.

According to still further features in the described preferred embodiments the at least one locus is selected from the group consisting of a biallelically expressed locus, a monoallelically expressed locus and a non-coding locus.

According to still further features in the described preferred embodiments the at least one locus is a biallelically expressed locus and/or a non-coding locus.

According to still further features in the described preferred embodiments the biallelically expressed locus comprises a tumor-suppressor gene, an oncogene, a transcription factor and a housekeeping gene.

According to still further features in the described preferred embodiments the biallelically expressed locus comprises a gene selected from the group consisting of HER2, CMYC, TP53, RB1, TP53, AML1, STS and KAL1.

According to still further features in the described preferred embodiments the monoallelically expressed locus comprises an imprinted gene, a gene subjected to chromosome X inactivation and/or random monoallelically expressed autosomal gene.

According to still further features in the described preferred embodiments the imprinted gene is selected from the group consisting of SNRPN, GRB10, GABRB3, UBE3A, IGF2, H19, CDKN1C and IGF2R.

According to still further features in the described preferred embodiments the gene subjected to chromosome X inactivation is XIST.

According to still further features in the described preferred embodiments the random monoallelically expressed autosomal gene is an odorant receptor gene, an interleukin gene and an immunoglobulin gene.

According to still further features in the described preferred embodiments the monoallelically expressed locus is selected from the group consisting of 15q11-13 and 11p15.

According to still further features in the described preferred embodiments the non-coding locus comprises a nucleic acid sequence associated with chromosome segregation.

According to still further features in the described preferred embodiments the nucleic acid sequence associated with chromosome segregation comprises alpha II satellite DNA and/or alpha III satellite DNA.

According to still further features in the described preferred embodiments the nucleic acid sequence associated with chromosome segregation is derived from CEN17, CEN15, CEN11 and CEN10.

According to still further features in the described preferred embodiments the cell of the individual is derived from blood.

According to still further features in the described preferred embodiments the cell of the individual is derived from bone marrow, urine, saliva and skin.

According to still further features in the described preferred embodiments the cell is derived from an unaffected tissue.

According to still further features in the described preferred embodiments the cell is a non-malignant cell.

According to still further features in the described preferred embodiments the cell is a malignant cell.

According to still further features in the described preferred embodiments the malignant cell is derived from a solid tumor, lymphoma, bone marrow and/or blood.

According to still further features in the described preferred embodiments the cell of the conceptus is derived from amniotic fluid, CVS, cord blood and placenta.

According to still further features in the described preferred embodiments the maternal cell is a blood cell.

According to still further features in the described preferred embodiments the maternal cell is derived from bone marrow, urine, saliva and skin.

According to still further features in the described preferred embodiments the cancer comprises a solid tumor.

According to still further features in the described preferred embodiments the chromosomal imbalance is gain of a whole chromosome or a portion thereof, loss of a whole chromosome or a portion thereof, duplication, deletion, microdeletion and imbalanced rearrangement.

According to still further features in the described preferred embodiments the chromosomal imbalance is Down syndrome, Turner syndrome, Edwards' syndrome, Patau's syndrome, Di-George syndrome, Prader-Willi syndrome (PWS), Angelman syndrome (AS), Beckwith-Wiedemann syndrome (BWS), Williams syndrome (WS) and/or Duchenne muscular dystrophy (DMD).

According to still further features in the described preferred embodiments the imprinting-associated chromosomal imbalance is Prader-Willi syndrome (PWS), Angelman syndrome (AS) and Beckwith-Wiedemann syndrome (BWS).

According to still further features in the described preferred embodiments the chromosomal imbalance is Down syndrome, Turner syndrome, Edwards' syndrome, Patau's syndrome, Di-George syndrome, Williams syndrome (WS) and/or Duchenne muscular dystrophy (DMD).

According to still further features in the described preferred embodiments the DNA methylation is detected by: (i) restriction enzyme digestion methylation detection; (ii) bisulphate-based methylation detection; (iii) mass-spectrometry analysis; (iv) sequence analysis; (v) microarray analysis; (vi) methylation density assay; and/or (vii) immunoprecipitation of methylated sequences.

According to still further features in the described preferred embodiments the chromatin methylation and the chromatin acetylation are detected by a decondensation assay.

According to still further features in the described preferred embodiments the replication pattern is detected by FISH.

According to still further features in the described preferred embodiments the at least one locus comprises a plurality of loci.

The present invention successfully addresses the shortcomings of the presently known configurations by providing methods of diagnosing cancer and of prenatally identifying a conceptus with imbalanced chromosome(s) using alterations in the interallelic epigenetic patterns and/or the replication patterns.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are histograms depicting the percentage of SD cells for the SNRPN imprinted locus assigned to 15q11-q13 in control samples (designated C1-C10) derived from subjects with normal karyotypes (unaffected individuals, control samples) (FIG. 1a) and patients with DiGeorge syndrome (designated D1-D10) (FIG. 1b). Each patient revealed the 22q11.2 deletion, characteristic for the DiGeorge syndrome. The last bar in each frame shows the mean value for the corresponding group of samples; the difference between the two groups is highly significant (P<10⁻⁹; Student's t-test). Note: while, control samples show high SD values, as expected for an imprinted locus, samples carrying a deletion reveal low SD values as expected from a biallelically expressed locus;

FIGS. 2a-c are histograms depicting the percentage of SD cells for the SNRPN imprinted locus assigned to 15q11-q13 in samples of patients with Velo-Cardio-Facial syndrome/DiGeorge syndrome (VCFS/DIGS; designated V1-V5 and V11-V15), each patient revealed the syndrome characteristic 22q11.2 deletion (FIG. 2a); samples of patients with Williams syndrome (WS; designated W1-W10), each patient revealed the syndrome characteristic 7q11.23 deletion (FIG. 2b) and control samples, derived from subjects with normal karyotypes (designated N16-N25) (FIG. 2c). The last bar in each frame shows the mean value for the corresponding group of samples. The two groups of patients show similar (P>0.05) and low SD values, not characteristic for an imprinted locus, each deviates significantly from the control group (P<10⁻⁹ for the VCFS/DIGS and P<10⁻⁷ for the WS; Student's t-test), which demonstrated high SD values, characteristic for an imprinted locus.

FIGS. 3a-c are histograms depicting the percentage of SD cells for the RB1 locus assigned to 13q24 in control samples, derived from subjects with normal karyotypes (designated N-N15) (FIG. 3a); samples of patients with Velo-Cardio-Facial syndrome/DiGeorge syndrome (VCFS/DIGS; designated V1-V10), each patient revealed the syndrome characteristic 22q11.2 deletion (FIG. 3b); and samples of patients with Williams syndrome (WS; designated W1-W10), each patient revealed the syndrome characteristic 7q11.23 deletion (FIG. 3c); The last bar in each frame shows the mean value for the corresponding group of samples. The group of control subjects revealed low SD values as expected for a biallelically expressed locus. In contrast, the two groups of patients displayed both similar (P>0.80) and high SD values. Each group of patients deviates significantly from the control group (P<10⁻⁶ for the VCFS/DIGS and P<10⁻⁴ for the WS; Student's t-test).

FIG. 4 is a histogram depicting the mean percentage of SD cells for the ARSA locus assigned to the long arm (q) of chromosome 22 distal to 22q11.2 in five control samples derived from subjects with normal karyotypes (controls) and ten samples of patients with Velo-Cardio-Facial syndrome/DiGeorge syndrome (VCFS/DIGS). Each patient revealed the 22q11.2 deletion, characteristic for the syndrome. The difference between the two groups is significant (P<0.0005; Student's t-test). Note: while, control samples show low SD values, as expected for a biallelically expressed locus, samples carrying a deletion reveal higher SD values, similar to a monoallelically expressed locus.

### DESCRIPTION OF THE PREFERRED EMBODIMENTS

The present invention is of interallelic epigenetic alterations in cells of cancer-stricken individuals which can be used for diagnosing cancer or cancer risk. In addition, the present invention is of interallelic epigenetic alterations and/or interallelic replication alterations in cells of a conceptus having imbalanced chromosome(s) or in maternal cells of the pregnant female carrying the conceptus, which can be used for prenatal diagnosis of chromosomal imbalances in the conceptus. Moreover, the present invention is of interallelic epigenetic alterations and/or interallelic replication alterations in cells of individuals having a chromosomal imbalance mosaicism which can be used for diagnosing chromosomal imbalance mosaicism, either prenatally or after birth (e.g., in a child or adult).

The principles and operation of the methods of diagnosing cancer or prenatally diagnosing a conceptus with imbalanced chromosome(s) according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Current diagnosis of cancer is based on invasive sampling of cancerous cells.

However, in many cases, diagnosis is enabled only in advanced stages of the cancer, e.g., when the cancer has spread. Thus, early diagnosis of cancer using non-invasive methods is highly desired.

Studies have demonstrated pathological methylation of certain cancer-related genes such as the mutL homolog 1 (MLH1) gene in cancerous cells (Kane et al., 1997; Baylin and Herman, 2000; Jones and Baylin, 2002). In addition, inhibitors of DNA methylation were found capable of reactivating the expression of genes, such as p16, that have undergone epigenetic silencing (Baylin S, et al., 2000).

The present inventors have previously disclosed a simple and non-invasive method of detecting cancer and cancer risk based on the level of asynchronous replication of alleles in blood samples (PCT Publication No. WO02/023187 A2 and U.S. Pat. No. 0,680,3195 to the present inventor).

However, although epigenetic changes were documented in cancer cells, none of these studies teaches diagnosing cancer by epigenetic analysis of non-malignant cells or even of malignant cell using loci which have not been associated to date with disease onset or progression.

While reducing the present invention to practice, and as is shown in Example 1 of the Examples section which follows, the present inventor has uncovered a method of diagnosing cancer by analyzing the interallelic epigenetic pattern.

Thus, according to one aspect of the present invention there is provided a method of diagnosing cancer.

As used herein the phrase “diagnosing cancer” refers to determining a presence or absence, classifying, determining a severity, monitoring disease progression, forecasting an outcome and/or prospects of recovery of the cancer. The term “diagnosing” also encompasses determining predisposition of an individual to become affected with cancer (e.g., the likelihood of an individual to develop cancer).

The cancer which is diagnosed by the method of this aspect of the present invention can be a solid tumor cancer (or carcinoma) or a nonsolid systemic cancer disease. Non-limiting examples of solid tumor cancers which can be diagnosed using the teachings of the present invention include breast cancer, prostate cancer, renal cancer, thyroid cancer, brain cancer, esophagus cancer, colon and colorectal cancer, liver cancer, pancreatic cancer, ovarian cancer, lung cancer, testicular cancer, osteosarcoma, skin cancer and bladder cancer. Non-limiting examples of nonsolid systemic cancer include leukemia, lymphoma, multiple myeloma, and myelodysplastic and myeloproliferative disorder syndromes.

Diagnosis of cancer according to the present invention is effected by determining in at least one locus of a cell of an individual in need thereof an interallelic epigenetic pattern, wherein an alteration in the interallelic epigenetic pattern compared to the interallelic epigenetic pattern of the at least one locus in a cell of an unaffected individual is indicative of the cancer.

The phrase “individual in need thereof” as used herein refers to a mammal, preferably a human being at any age which is suspected of having cancer, is predisposed to develop cancer [e.g., a carrier of a mutation in a double-strand-break (DSB) checkpoint/repair genes such as ATM, BRCA1 and TP53] or is affected with cancer.

The phrase “unaffected individual” as used herein refers to a mammal individual as above who is not affected, predisposed or suspected to have the cancer.

The phrase “interallelic epigenetic pattern” as used herein refers to the epigenetic state of each of the alleles present in a certain locus resulting from presence or absence of an epigenetic modification at a specific position. The epigenetic state of the alleles in a certain locus can be determined by the state of DNA methylation of the cytosine residue of a CpG (cytosine-phosphate-guanine) dinucleotide and the state of chromatin epigenetic modification which alters chromatin conformation. The term “chromatin” refers to DNA condensed with proteins, mainly histones. The phrase “chromatin conformation” as used herein refers to the status of chromatin packing, either tight or loose which depends on histone methylation (e.g., methylation of a lysine residue at position 9 on the N-terminus of histone H3 or of a lysine residue at position 4 of histone H3) and/or histone acetylation on conserved lysine residues.

According to the method of this aspect of the present invention the interallelic epigenetic state is determined in at least one locus of a cell. The term “locus” as used herein refers to a defined location on a chromosome. Preferably the locus used by the present invention includes coding nucleic acid sequence (e.g., a gene) or non-coding nucleic acid (DNA) sequences. The term “gene” as used herein refers to a nucleic acid sequence with a transcriptional capability, i.e., which can be transcribed into an RNA sequence (an expressed sequence) which in most cases, is translated into an amino acid sequence, along with the regulatory sequences that regulate expression or engage in the expression of expressed sequences.

The locus in which the interallelic epigenetic pattern is determined can be a monoallelically expressed locus, a biallelically expressed locus and/or a non-coding locus.

Biallelic expression refers to an expression state of a locus and/or a specific gene wherein both alleles are expressed about equally. It will be appreciated that certain genes may have a tissue or cell type-specific mode of expression, namely, they may have a biallelic expression in one type of tissue or cell and a monoallelic expression in another type of tissue or cell. A non-limiting example of such a gene is the UBE3A gene (also known as E6AP), which is monoallelically expressed in certain brain cells such as in the cerebellum and is biallelically expressed in other cells such as blood lymphocytes. Thus, the phrase “biallelically expressed locus” refers to the mode of expression in the tested (analyzed) cell or tissue.

The biallelically expressed loci which are used by the method of this aspect of the present invention can be from any loci in which both alleles are equally expressed in the cell type used by the present invention. Such a locus can include, for example, a tumor-suppressor gene, an oncogene, a transcription factor and/or a housekeeping gene. Non-limiting examples of biallelically expressed loci are those including the genes coding HER2 (GenBank Accession Nos. NM_{—}00100586, NM_{—}004448.2), CMYC (GenBank Accession No. NM_{—}002467.3), TP53 (GenBank Accession No. NM_{—}000546.2), RB1 (GenBank Accession No. NM_{—}000321.1) and AML1 (RUNX1; GenBank Accession No. NM_{—}001754.3, NM_{—}001001890.1), STS (steroid sulfatase; GenBank Accession No. NM_{—}000351.3), KAL1 (GenBank Accession No. NM_{—}000216.1), beta-actin (GenBank Accession No. NM_{—}001101.2) and GAPDH (GenBank Accession No. NM_{—}002046.2).

Monoallelic expression refers to an expression state of a locus and/or a gene wherein one allele is expressed at a significantly lower level compared to the other, for instance, when one allele is silent (i.e., not expressed) and the other allele is active (i.e., expressed).

A monoallelically expressed gene can be an imprinted gene such as SNRPN (GenBank Accession Nos. NW_{—}925783 (contig), NM_{—}022807.2, NM_{—}022808.2, NM_{—}022806.2, NM_{—}022805.2 and NM_{—}003097.3), GRB10 (GenBank Accession No. NM_{—}001001556.1, NM_{—}001001549.1, NM_{—}005311.3, NM_{—}001001550.1), GABRB3 (GenBank Accession No. NM_{—}021912.2, NM_{—}000814.3), UBE3A (D15S10; GenBank Accession No. NM_{—}130839.1, NM_{—}000462.2, NM_{—}130838.1), IGF2 (GenBank Accession No. NM_{—}000612.2), H19 (GenBank Accession No. NR_{—}002196.1), CDKN1C (GenBank Accession No. NM_{—}000076.1) and IGF2R (GenBank Accession No. NM_{—}000876.1).

Additionally or alternatively, a monoallelically expressed gene can be a gene located on the X chromosome which is subjected to chromosome X inactivation on the female genome such as XIST (GenBank Accession No. NR_{—}001564.1).

Still additionally or alternatively, a monoallelically expressed gene can be a random monoallelically expressed autosomal gene (also referred to as gene undergoing allelic exclusion) such as an odorant receptor gene [e.g., OR1J4 (GenBank Accession No. NM_{—}00100445), OR2AT4 (GenBank Accession No. NM_{—}00100528), OR4F15 (GenBank Accession No. NM_{—}00100167), OR4X2 (GenBank Accession No. NM_{—}00100472), OR6B3 (GenBank Accession No. NM_{—}173351.1), OR7D2 (GenBank Accession No. NM_{—}175883.1), OR10A3 (GenBank Accession No. NM_{—}00100374), OR13C4 (GenBank Accession No. NM_{—}00100191)], an interleukin gene [e.g., IL1F9 (GenBank Accession No. NM_{—}019618), IL5 (GenBank Accession No. NM_{—}000879.2), IL12B (GenBank Accession No. NM_{—}002187.2), IL16 (GenBank Accession No. NM_{—}172217.1), IL17B (GenBank Accession No. NM_{—}014443.2)], an immunoglobulin gene [e.g., K-immunoglobulin gene (IGK; GenBank Accession No. NG_{—}000834, NG_{—}000833)] and a T-cell receptor gene. For further details on random monoallelically expressed autosomal genes see Ensminger A W and Chess A, 2004, Hum. Mol. Genet. 13: 651-658 and Singh N., et al., 2003, Nat. Genet. 33:1-3, which are fully incorporated herein by reference.

Non-limiting examples of monoallelically expressed loci include the PWS-AS locus on chromosome 15q11-13 and the Beckwith-Wiedemann syndrome imprinted region on chromosome 11p15.

The phrase “non-coding DNA” refers to a nucleic acid sequence lacking transcriptional capability. Preferably, the non-coding locus includes a nucleic acid sequence associated with chromosome segregation such as alpha II satellite DNA and/or alpha III satellite DNA such as those derived from the centromere of chromosome 17 (CEN17; D17Z1), the centromere of chromosome (CEN15; D15Z1), the centromere of chromosome 11 (CEN11; D11Z1) and the centromere of chromosome 10 (CEN10; DZ10).

Preferably, the method of this aspect of the present invention contemplates the use of a plurality of loci, i.e., more than one locus, preferably two loci, more preferably, three loci, more preferably, four loci, even more preferably, more than five loci. The plurality of loci can be selected such that one (single) or more loci are monoallelically expressed, one or more loci are biallelically expressed, and/or one (single) or more loci are non-coding. Alternatively, the plurality of loci can be selected all from one class of loci (e.g., monoallelically expressed loci) or from any combination of loci from monoallelically expressed, biallelically expressed and/or non-coding loci.

The cell of this aspect of the present invention can be any cell which is derived from the individual. Examples include, but are not limited to, cells derived from a biological sample such as blood, saliva, urine, excrement, a tissue biopsy such as bone marrow, skin, liver, spleen, kidney, heart and lymph node. Such cells can be obtained using methods known in the art, including, but not limited to, blood drawing, fine needle biopsy, needle biopsy, core needle biopsy and surgical biopsy (e.g., brain or liver biopsy).

As described in Example 1 of the Examples section which follows, the method of diagnosing cancer according to the present invention can utilize malignant cells (e.g., which are derived by a biopsy of a suspected cancerous tissue, a solid tumor, a lymphoma, bone marrow or blood) as well as non-malignant cells (which are derived from an unaffected tissue, i.e., a tissue devoid of cancer and not suspected to have cancerous cells). For example, in the diagnosis of prostate cancer or breast cancer, the malignant cells can be derived from the prostate or breast tissue, respectively, and the non-malignant cells can be derived from peripheral blood or skin (e.g., a skin biopsy taken from the arm).

The cell used by the method of this aspect of the present invention can be an uncultured cell (as obtained from the individual) or can be cultured along with the appropriate tissue culture medium (a cell culture). It will be appreciated that some cells, when subject to culturing conditions in the presence of a tissue culture medium can continue their mitotic cell division without any specific additives (e.g., growth factors) to the medium. Non-limiting examples of such cells include fibroblasts obtained from a skin biopsy. On the other hand, to induce mitotic division of other cells such as blood lymphocytes, the cells are preferably cultured in the presence of a mitotic division stimulating agent such as phytohemagglutinin (PHA).

Regardless of the growth conditions employed, the cells are preferably cultured for a time period sufficient to enable at least one population doubling of the cells and to avoid epigenetic modifications. As used herein the phrase “population doubling” refers to a two-fold increase in the total number of cells in a culture, most commonly during the exponential, or “log”, phase of growth. Preferably, the cells are cultured for a time period sufficient for at least two population doublings, more preferably, at least three population doublings. For example, in case blood lymphocytes are used, the cells are preferably cultured for at least 24 hours, more preferably, at least 36 hours, more preferably, at least 48 hours, even more preferably, at least 72 hours.

As described in Example 1 of the Examples section which follows, in order to discriminate between inter-individual epigenetic polymorphism and/or pathological epigenetic patterns which are related to cancer and not to inherited epigenetic, a portion of the cells is cultured in the presence of an epigenetic modifier agent.

As used herein, the phrase “epigenetic modifier agent” refers to an agent capable of modifying the conformation of the chromatin and/or the methylation state of the DNA. Factors known to affect the chromatin packing include histone methylation, demethylation, acetylation and deacetylation. Thus, the epigenetic modifier agent used by the present invention is capable of modifying the methylation state of the DNA or of the histone(s) and/or the acetylation state of histone(s). Preferably, the epigenetic modifier agent used by the method of the present invention is capable of reversing a pathological epigenetic alteration such as that caused from the presence of cancer cells and/or genomes with imbalanced chromosome(s).

Preferably, the methylation modifying agent is a DNA methylation inhibitor such as 5-azacytidine (5-aza-CR), 5-aza-2′deoxycytidine (5-aza-CdR), 5 fluorocytosine, pseudoisocytosine, Zebularine, Procainamide, polyphenol (−)-epigallocatechin-3-gallate (EGCG), and Psammaplin; hi stone methylation inhibitor and/or histone demethylation inhibitor. Preferably, the acetylation modifying agent is a histone deacetylase (HDAC) inhibitor such as Trichostatin A (TSA), Sodium butyrate, suberoylanilide hydroxamic acid (SAHA) and N-nitroso-n-methylurea; a histone acetyltransferase (HAT) inhibitor such as Polyisoprenylated Benzophenone (Garcinol) and Set/TAF-1 beta; histone deacetylase and histone acetyltransferase. Such inhibitors and enzymes can be purchased from InvivoGen, Errant Gene Therapeutics and Aton Pharma. It should be noted that DNA methylation inhibitors such as 5-aza-CR and 5-aza-CdR are converted to the deoxynucleotide triphosphates and are then incorporated in place of cytosine into replicating DNA. They are therefore active only in S-phase cells, where they serve as powerful mechanism-based inhibitors of DNA methylation.

Thus, the method according to this aspect of the present invention contemplates the use of uncultured cells, cells cultured in the presence of an epigenetic modifier agent and/or cells cultured in the absence of the epigenetic modifier agent. Alterations in the interallelic epigenetic pattern can be determined by comparing the interallelic epigenetic pattern of uncultured cells between various individuals (e.g., the individual in need thereof and an unaffected individual), as well as the interallelic epigenetic pattern of cultured cells (in the presence and/or absence of an epigenetic modifier agent) of various individuals. In addition, as described in the Examples section which follows, alterations in the interallelic epigenetic pattern can be determined by comparing the interallelic epigenetic pattern of uncultured cells, cultured cells in the absence of the epigenetic modifier agent and/or cultured cells in the presence of the epigenetic modifier agent, of either the same individual or between different individuals (e.g., the individual in need thereof and an unaffected individual). The latter approach is capable of discriminating between natural, polymorphism(s) present between individuals of a population and between interallelic, epigenetic modifications which occur as a result of a pathological condition such as the presence of cancer or of imbalanced chromosomes in cells of the individual. Thus, natural polymorphisms associated with interallelic epigenetic modifications can be present in uncultured cells. However, to verify that such modifications are not due to the presence of cancer and/or imbalanced chromosome(s) of the individual, the cells are preferably cultured in the presence or absence of the epigenetic modifier agent. A modification in the interallelic epigenetic pattern between cells cultured in the presence of the epigenetic modifier agent (e.g., 5-aza-CR) and cells cultured in the absence of the epigenetic modifier agent is indicative for the presence of cancer. Alternatively, if the interallelic epigenetic pattern is not altered as a result of culturing in the presence of the epigenetic modifier agent (as compared to culturing in the absence of the epigenetic modifier agent), then the alteration between the individuals is due to a polymorphism (e.g., an inherited polymorphism) between the individuals.

A number of approaches for determining interallelic DNA methylation are known in the art including restriction enzyme digestion-based methylation detection and bisulphate-based methylation detection. Several such approaches are summarized infra and in the Example 1 of the Examples section which follows [further details on techniques useful for detecting methylation are disclosed in Ahrendt (1999) J. Natl. Cancer Inst. 91:332-9; Belinsky (1998) Proc. Natl. Acad. Sci. USA 95:11891-96; Clark (1994) Nucleic Acids Res. 22:2990-7; Herman (1996) Proc. Natl. Acad. Sci. USA 93:9821-26; Xiong and Laird (1997) Nuc. Acids Res. 25:2532-2534].

Restriction enzyme digestion methylation detection assay is based on the inability of some restriction enzymes to cut methylated DNA. Typically used are the enzyme pairs HpaII-MspI including the recognition motif CCGG, and SmaI-XmaI with a less frequent recognition motif, CCCGGG. Thus, for example, HpaII is unable to cut DNA when the internal cytosine in methylated, rendering HpaII-MspI a valuable tool for rapid methylation analysis. The method is usually performed in conjunction with a Southern blot analysis. Measures are taken to analyze a gene sequence which will not give a difficult to interpret result. Thus, a region of interest flanked with restriction sites for CG methylation insensitive enzymes (e.g., BamHI) is first selected. Such sequence is selected not to include more than 5-6 sites for HpaII. The probe(s) used for Southern blotting or PCR should be located within this region and cover it completely or partially. This method has been successfully employed by Buller and co-workers (1999) Association between nonrandom X-chromosome inactivation and BRCA1 mutation in germline DNA of patients with ovarian cancer J. Natl. Cancer Inst. 91(4):339-46. Since digestion by methylation sensitive enzymes (e.g., HpaII) is often partial, a complementary analysis with McrBC or other enzymes which digest only methylated CpG sites is preferable [Yamada et al. Genome Research 14 247-266 2004] to detect various methylation patterns.

Bisulphate-based methylation genomic sequencing is described in Clark et al., (1994) supra, and is capable of detecting every methylated cytosine on both strands of any target sequence, using DNA isolated from fewer than 100 cells. In this method, sodium bisulphite is used to convert cytosine residues to uracil residues in single-stranded DNA, under conditions whereby 5-methylcytosine remains non-reactive. The converted DNA is amplified with specific primers and sequenced. All the cytosine residues remaining in the sequence represent previously methylated cytosines in the genome. This method utilizes defined procedures that maximize the efficiency of denaturation, bisulphite conversion and amplification, to permit methylation mapping of single genes from small amounts of genomic DNA, readily available from germ cells and early developmental stages.

Methylation-specific PCR (MSP) is the most widely used assay for the sensitive detection of methylation. Briefly, prior to amplification, the DNA is treated with sodium bisulphite to convert all unmethylated cytosines to uracils. The bisulphite reaction effectively converts methylation information into sequence difference. The DNA is amplified using primers that match one particular methylation state of the DNA, such as that in which DNA is methylated at all CpGs. If this methylation state is present in the DNA sample, the generated PCR product can be visualized on a gel. It will be appreciated, though, that the method specific priming requires all CpG in the primer binding sites to be co-methylated. Thus, when there is comethylation, an amplified product is observed on the gel. When one or more of the CpGs is unmethylated, there is no product. Therefore, the method does not allow discrimination between partial levels of methylation and complete lack of methylation [See U.S. Pat. No. 5,786,146; Herman et al., Proc. Natl. Acad. Sci. USA 93: 9821-9826 (1996)].

Real-time fluorescent MSP (MethyLight) is based on real time PCR employing fluorescent probes in conjunction with MSP and allows for a homogeneous reaction which is of higher throughput. If the probe does not contain CpGs, the reaction is essentially a quantitative version of MSP. However, the fluorescent probe is typically designed to anneal to a site containing one or more CpGs, and this third oligonucleotide increases the specificity of the assay for completely methylated target strands. Because the detection of the amplification occurs in real time, there is no need for a secondary electrophoresis step. Since there is no post PCR manipulation of the sample, the risk of contamination is reduced. The MethyLight probe can be of any format including but not limited to a Taqman probe or a LightCycler hybridization probe pair and if multiple reporter dyes are used, several probes can be performed simultaneously [Eads (1999) Cancer Res. 59:2302-2306; Eads (2000) Nucleic Acids Res. 28:E32; Lo (1999) Cancer Res. 59:3899-390]. The advantage of quantitative analysis by MethyLight was demonstrated with glutathione-S-transferase-P1 (GSTP1) methylation in prostate cancer [Jeronimo (2001) J. Natl. Cancer Inst. 93:1747-1752]. Using this method it was possible to show methylation in benign prostatic hyperplasia samples, prostatic intraexpithelial neoplasma regions and localized prostate adenocarcinoma.

Methylation density assay is a quantitative method for rapidly assessing the CpG methylation density of a DNA region as previously described by Galm et al. (2002) Genome Res. 12, 153-7. Basically, after bisulfite modification of genomic DNA, the region of interest is PCR amplified with nested primers. PCR products are purified and DNA amount is determined. A predetermined amount of DNA is incubated with ³H-SAM (TRK581Bioscience, Amersham) and SssI methyltransferase (M0226, New England Biolabs Beverly, Mass. 01915-5599, USA) for methylation quantification. Once reactions are terminated products are purified from the in-vitro methylation mixture. 20% of the eluant volume is counted in ³H counter. Normalizing radioactivity DNA of each sample is measured again and the count is normalized to the DNA amount.

Restriction analysis of bisulphite modified DNA is a quantitative technique also called COBRA (Xiong and Laird, 1997, Nuc. Acids Res. 25:2532-2534) which can be used to determine DNA methylation levels at specific gene loci in small amounts of genomic DNA. Restriction enzyme digestion is used to reveal methylation-dependent sequence differences in PCR products of sodium bisulfite-treated DNA. Methylation levels in original DNA sample are represented by the relative amounts of digested and undigested PCR product in a linearly quantitative fashion across a wide spectrum of DNA methylation levels. This technique can be reliably applied to DNA obtained from microdissected paraffin-embedded tissue samples. COBRA thus combines the powerful features of ease of use, quantitative accuracy, and compatibility with paraffin sections.

Differential methylation hybridization (DMH) integrates a high-density, microarray-based screening strategy to detect the presence or absence of methylated CpG dinucleotide genomic fragments [See Schena et al., Science 270: 467-470 (1995)]. Array-based techniques are used when a number (e.g., >3) of methylation sites in a single region are to be analyzed. First, CpG dinucleotide nucleic acid fragments from a genomic library are generated, amplified and affixed on a solid support to create a CpG dinucleotide rich screening array. Amplicons are generated by digesting DNA from a sample with restriction endonucleases which digest the DNA into fragments but leaves the methylated CpG islands intact. These amplicons are used to probe the CpG dinucleotide rich fragments affixed on the screening array to identify methylation patterns in the CpG dinucleotide rich regions of the DNA sample. Unlike other methylation analysis methods such as Southern hybridization, bisulfite DNA sequencing and methylation-specific PCR which are restricted to analyzing one gene at a time, DMH utilizes numerous CpG dinucleotide rich genomic fragments specifically designed to allow simultaneous analysis of multiple of methylation-associated genes in the genome (for further details see U.S. Pat. No. 6,605,432).

Immunoprecipitation of methylated sequences can be used to isolate sequence-specific methylated DNA fragments. Briefly, genomic DNA is sonicated to yield fragments of 200-300 bp. The DNA is then denatured, precleaned with a protein A Fast FlowSepharose (Amersham Biosciences) and further incubated with a 5-methylcytidine monoclonal antibody (Eurogenetc). The complex is purified using protein A Sepharose (Pharmacia) and washed. The immunoprecipitated samples are analyzed using specific PCR primers, essentially as described in Mukhopadhyay R., et al., Genome Research 14: 1594-1602.

Further details and additional procedures for analyzing DNA methylation (e.g., mass-spectrometry analysis) are available in Tost J, Schatz P, Schuster M, Berlin K, Gut I G. Analysis and accurate quantification of CpG methylation by MALDI mass spectrometry. Nucleic Acids Res. 2003 May 1; 31(9):e50; Novik K L, Nimmrich I, Genc B, Maier S, Piepenbrock C, Olek A, Beck S. Epigenomics: genome-wide study of methylation phenomena. Curr Issues Mol. Biol. 2002 October; 4(4):111-28. Review; Beck S, Olek A, Walter J. From genomics to epigenomics: a loftier view of life. Nat. Biotechnol. 1999 December; 17(12):1144; Fan (2002) Oncology Reports 9:181-183; http://www.methods-online.net/methods/DNAmethylation.html; Shi (2003) J. Cell Biochem. 88(1):138-43; Adoryian (2002) Nucleic Acids Res. 30(5):e21.

It will be appreciated that a number of commercially available kits may be used to detect the interallelic methylation state of the locus of the present invention. Examples include, but are not limited to, the EZ DNA methylation Kit™ (available from Zymo Research, 625 W Katella Ave, Orange, Calif. 92867, USA). Typically, oligonucleotides for the bisulphate-based methylation detection methods described hereinabove are designed according to the technique selected.

As used herein the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions (see disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050).

Thus, for example, the most critical parameter affecting the specificity of methylation-specific PCR is determined by primer design. Since modification of DNA by bisulfite destroys strand complementarity, either strand can serve as the template for subsequent PCR amplification, and the methylation pattern of each strand can then be determined. It will be appreciated, though, that amplifying a single strand (e.g., sense) is preferable in practice. Primers are designed to amplify a region that is 80-250 bp in length, which incorporates enough cytosines in the original strand to assure that unmodified DNA does not serve as a template for the primers. In addition, the number and position of cytosines within the CpG dinucleotide determines the specificity of the primers for methylated and unmethylated templates. Typically, 1-3 CpG sites are included in each primer and concentrated in the 3′ region of each primer. This provides optimal specificity and minimizes false positives due to mispriming. To facilitate simultaneous analysis of each of the primers of a given gene in the same thermocycler, the length of the primers is adjusted to give nearly equal melting/annealing temperatures.

Furthermore, since MSP utilizes specific primer recognition to discriminate between methylated and unmethylated alleles, stringent annealing conditions are maintained during amplification. Essentially, annealing temperatures is selected maximal to allow annealing and subsequent amplification. Preferably, primers are designed with an annealing temperature 5-8 degrees below the calculated melting temperature. For further details see Herman J. G. and Baylin S. B. (1998) Methylation-Specific PCR. In: Current Protocols in Human Genetics. Dracopoli N. C. et al. (eds), Unit 10.6. Copy right 2003 John Willey & Sons, Inc.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The interallelic chromatin methylation and/or acetylation state can be detected by the evaluating the condensation/decondensation state of the chromatin using various approaches. For example, as described in Example 1 of the Example section which follows, nuclei can be permeabilized with Triton X-100 and then subjected to digestion with micrococcal nuclease (MNase). At predetermined time points the reaction is stopped (e.g., by the addition of proteinase K) and the digested DNA is prepared, treated with RNase A, resolved on an agarose gel and stained with ethidium bromide. Alternatively, the decondensation state of the chromatin can be evaluated by detecting the release of histones from the chromatin following MNase digestion, essentially as described in Meshorer E., et al., 2006, Developmental Cell, 10: 105-116. Briefly, nuclei are subject to MNase digestion (1 U/ml, Worthington, Lakewood, N.J.) in 10 mM Tris-HCl buffer supplemented with 5 mM CaCl₂. The reactions are centrifuged for 10 minutes at 14,000×g and the supernatants are collected and run on an SDS gel (e.g., 4-20% Tris-HCl SDS gel, BioRad). Still alternatively, the decondensation state of the chromatin can be detected by subjecting cells (e.g., in interphase) to immunostaining with antibodies against tetra-acetylated H4, lysine 4-di-methylated H3 or lysine 9-di-methylated H3 followed by counterstaining with DAPI, essentially as described in Probst A V., et al., 2003, The Plant Journal, 33: 743-749.

The interallelic epigenetic change can result from the interaction of small RNA molecules such as microRNA with genomic or RNA sequences and which leads to silencing of certain genes or of large chromosomal segments (e.g., chromosome X inactivation by the binding of XIST RNA). Such changes can be detected as described in He L. and Hannon G J. (2004) Nat. Rev. Genet. 5: 522-532; Matzke M A and Birchler J A (2005) Nat. Rev. Genet. 6: 24-35; Lippman Z., et al (2004) Nature 430: 471-476; Landthaler, M., et al., 2004, Current Biology, 14: 2162-2167; which are fully incorporated herein by reference.

Thus, the present invention contemplates a diagnostic test based on small aliquots of peripheral blood that identifies subjects with various types of solid tumors as well as hematological malignancies with a positive predicted value of about 90% or more. The test offers a decisive advantage for cancer detection. It not only prevents invasive procedures that are hazardous, painful and costly, but also enables earlier detection, which is crucial for effective treatment, and possible cure, of cancer. Moreover, it provides a reliable tool for the detection of a minimal residual malignant disease following completion of the therapy course.

It will be appreciated that the method according to this aspect of the present invention can be used for screening of cancer in a population. Individuals which can be screened according to the teachings of the present invention are those being at risk to develop cancer due to family history (e.g., individuals who have a first degree or second degree relative who is/was diagnosed with cancer), individuals predisposed to cancer due to an inheritance of a mutation in gene associated with increased predisposition to cancer (e.g., p53, BRCA1, BRCA2), individuals who are at risk to develop cancer due to occupational hazard (e.g., exposure to radiation such as ionizing radiation, cellular radiation, radio-isotopes), exposure to various carcinogens, cigarette smoking and the like, and/or individuals from a certain age or body weight (e.g., above 40 years, preferably, above 50 years) which have increased risk to develop cancer due to their age or weight.

Thus, screening of cancer using the teachings of the present invention can be easily performed in any laboratory, by drawing a blood sample (similar to PSA testing for prostate cancer) from the individual and determining alterations between the interallelic epigenetic pattern of the individual in need and an unaffected individual (e.g., a young, healthy individual, not predisposed to cancer, not subject to occupational hazard).

Prenatal diagnosis, the analysis of fetal cells, is currently performed using invasive methods such as amniocentesis, chorionic villus sampling (CVS) and cordocenthesis. Prenatal testing of fetal cells includes cytogenetic analysis (i.e., karyotype determination and FISH analysis) as well as gene-specific DNA analysis (e.g., for the identification of gene-specific diseases such as cystic fibrosis). However, in many cases, in spite of a comprehensive cytogenetic analysis, subtle chromosomal alterations such as micro-deletions (e.g., 22q11 causing Di-George syndrome) can be missed, resulting in the birth of an affected child. In addition, in several cases, in the absence of an affected sibling, detection of specific genetic aberrations such as those related to single-gene disorders (e.g., canavan, cystic fibrosis) or imprinting disorders (e.g., Angelman syndrome) is not routinely performed. Thus, there is a long felt need, and it is highly desired to develop method of prenatal diagnosis which provides a comprehensive analysis of all chromosomal imbalances, including those which can be missed by the routine cytogenetic analyses. In addition, due to the risk associated with invasive sampling of fetal cells, it is highly desirable to develop accurate non-invasive prenatal diagnosis methods.

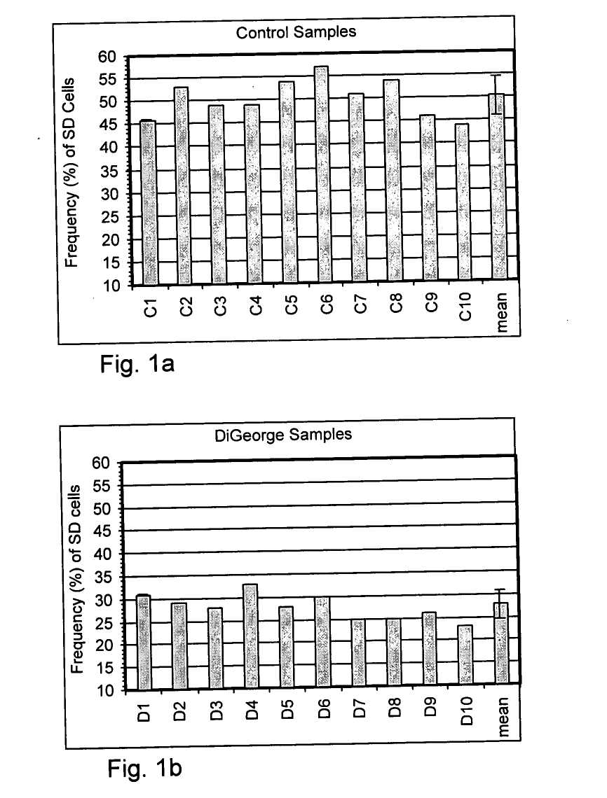

As is shown in FIGS. 1-4, described in Example 2 of the Examples section which follows and in the background section hereinabove, genomes with chromosomal imbalances such as trisomies, monosomies, micro deletions and/or small duplications may display alterations in the temporal order of allelic replication of genes not associated with the aberrant chromosome(s) (Amiel et al. 1998a, 1999; Goldshtein 2004; Senn 2003; Reish et al 2002; Ofir et al. 1999; Amiel at al. 2001, 2002). In addition, it was uncovered that co-culturing of normal (euploid) amniocytes with aneuploid amniocytes, derived from fetuses with either Down, Edwards, Patau or Turner syndromes, resulted in aberrant replication phenotype of the normal amniocytes (Goldshtein, 2004). Moreover, Senn et al. (2003), demonstrated that the loss of the inherent temporal order of allelic replication in the aneuploid genotypes can be reversed in the presence of 5-azacytidine, an inhibitor of DNA methylation.

However, although alterations in the replication pattern were documented in individuals with abnormal chromosomes, to date a method of prenatally diagnosing a fetus, which is based on determining alterations in the interallelic epigenetic pattern in cells of a conceptus or maternal cells of the pregnant female carrying the conceptus was not suggested or taught.

Thus, according to another aspect of the present invention there is provided a method of prenatally identifying a chromosomal imbalance in a conceptus.

As used herein the term “prenatally” refers to the period of time between the conception and the birth of an infant. The phrase “prenatal identifying” refers to the identification of chromosomal imbalances of a conceptus before birth.

The term “conceptus” as used herein refers to the product of conception at any point between fertilization and birth. The term “conceptus” includes an embryo, a fetus or an extraembryonic membrane of an ongoing pregnancy as well as of a terminated pregnancy (e.g., following miscarriage, abortion or delivery of a dead fetus).

The method is effected by determining in at least one locus of a maternal cell of a pregnant female carrying the conceptus an interallelic epigenetic pattern, wherein an alteration in the interallelic epigenetic pattern compared to the interallelic epigenetic pattern of the at least one locus in a cell of an individual devoid of the chromosomal imbalance is indicative of the chromosomal imbalance in the conceptus.

As used herein the phrase “maternal cell” refers to any cell which is derived from the pregnant female carrying the conceptus and which is subject to an alteration in the interallelic epigenetic pattern as a result of the presence of a conceptus with imbalanced chromosome(s) in its genome. Examples of maternal cells which can be used according to this aspect of the present invention include, but are not limited to cells derived from a biological sample of the pregnant female such as the maternal blood, bone marrow, urine, saliva and skin.

As used herein the phrase “chromosomal imbalance” refers to an abnormal number of chromosomes (e.g., gain or loss of a whole chromosome or a portion thereof), a chromosomal structure abnormality (e.g., a deletion including a macrodeletion and a microdeletion, duplication, imbalanced rearrangement such as imbalanced translocation and/or imbalanced inversion) and/or an epigenetic abnormality [e.g., abnormal methylation pattern on the DNA or the chromatin and/or an abnormal acetylation pattern of the chromatin].

According to preferred embodiments of the present invention, the chromosomal imbalance can be chromosomal aneuploidy [i.e., complete and/or partial multisomy (e.g., trisomy) and/or monosomy], imbalanced rearrangement such as imbalanced translocation or imbalanced inversion, deletion, macrodeletion, microdeletion and/or duplication (i.e., complete an/or partial chromosome duplication).

The phrase “imbalanced translocation” or “imbalanced inversion” refer to any translocation or inversion, respectively, which results in a loss or a gain of chromosome segment(s).

Non-limiting examples of trisomies or partial trisomies which can be detected by the present invention include trisomy 21, trisomy 18, trisomy 16, trisomy 13, XXY, XYY, XXX, partial trisomy 1q32-44 (Kimya Y et al., Prenat Diagn. 2002, 22:957-61), trisomy 9p with trisomy 10p (Hengstschlager M et al., Fetal Diagn Ther. 2002, 17:243-6), trisomy 4 mosaicism (Zaslav A L et al., Am J Med. Genet. 2000, 95:381-4), trisomy 17p (De Pater J M et al., Genet Couns. 2000, 11:241-7), partial trisomy 4q26-qter (Petek E et al., Prenat Diagn. 2000, 20:349-52), trisomy 9 (Van den Berg C et al., Prenat. Diagn. 1997, 17:933-40), partial 2p trisomy (Siffroi J P et al., Prenat Diagn. 1994, 14:1097-9), partial trisomy 1q (DuPont B R et al., Am J Med Genet. 1994, 50:21-7), and/or partial trisomy 6p/monosomy 6q (Wauters J G et al., Clin Genet. 1993, 44:262-9).

Non-limiting examples of monosomies which can be detected by the present invention include monosomy 22, 16, 21 and 15, which are known to be involved in pregnancy miscarriage (Munne, S. et al., 2004. Reprod Biomed Online. 8: 81-90)], monosomy X, monosomy 21, monosomy 22, monosomy 16 and monosomy 15.

Non-limiting examples of microdeletions which can be detected by the present invention include the 15q11-q13 microdeletion (associated with PWS-AS), 11q23 microdeletion (Matsubara K, Yura K. Rinsho Ketsueki. 2004, 45:61-5); Smith-Magenis syndrome 17p11.2 deletion (Potocki L et al., Genet Med. 2003, 5:430-4); 22q13.3 deletion (Chen C P et al., Prenat Diagn. 2003, 23:504-8); Xp22.3. microdeletion (Enright F et al., Pediatr Dermatol. 2003, 20:153-7); 10 p14 deletion (Bartsch O, et al., Am J Med. Genet. 2003, 117A:1-5); 20p microdeletion (Laufer-Cahana A, Am J Med. Genet. 2002, 112:190-3.), DiGeorge syndrome [del(22)(q11.2q11.23)], Williams syndrome [7q11.23 and 7q36 deletions, Wouters C H, et al., Am J Med. Genet. 2001, 102:261-5.]; 1p36 deletion (Zenker M, et al., Clin Dysmorphol. 2002, 11:43-8); 2p microdeletion (Dee S L et al., J Med. Genet. 2001, 38:E32); neurofibromatosis type 1 (17q11.2 microdeletion, Jenne D E, et al., Am J Hum Genet. 2001, 69:516-27); Yq deletion (Toth A, et al., Prenat Diagn. 2001, 21:253-5); Wolf-Hirschhorn syndrome (WHS, 4 p16.3 microdeletion, Rauch A et al., Am J Med. Genet. 2001, 99:338-42); 1p36.2 microdeletion (Finelli P, Am J Med Genet. 2001, 99:308-13); 11q14 deletion (Coupry I et al., J Med. Genet. 2001, 38:35-8); 19q13.2 microdeletion (Tentler D et al., J Med. Genet. 2000, 37:128-31); Rubinstein-Taybi (16 p13.3 microdeletion, Blough R I, et al., Am J Med. Genet. 2000, 90:29-34); 7p21 microdeletion (Johnson D et al., Am J Hum Genet. 1998, 63:1282-93); Miller-Dieker syndrome (17 p13.3), 17 p1.2 deletion (Juyal R C et al., Am J Hum Genet. 1996, 58:998-1007); 2q37 microdeletion (Wilson L C et al., Am J Hum Genet. 1995, 56:400-7).

Non-limiting examples of translocations which can be detected by the present invention include the t(11; 14)(p15; p13) translocation (Benzacken B et al., Prenat Diagn. 2001, 21:96-8); unbalanced translocation t(8; 11)(p23.2; p 15.5) (Fert-Ferrer S et al., Prenat Diagn. 2000, 20:511-5);

Non-limiting examples of inversions which can be detected by the present invention include the inverted chromosome X (Lepretre, F. et al., Cytogenet. Genome Res. 2003. 101: 124-129; Xu, W. et al., Am. J. Med. Genet. 2003. 120A: 434-436), inverted chromosome 10 (Helszer, Z., et al., 2003. J. Appl. Genet. 44: 225-229).

Other chromosomal imbalances which can be detected by the method of the present invention include mosaic for a small supernumerary marker chromosome (SMC) (Giardino D et al., Am J Med. Genet. 2002, 111:319-23), cryptic subtelomeric chromosome rearrangements (Engels, H., et al., 2003. Eur. J. Hum. Genet. 11: 643-651; Bocian, E., et al., 2004. Med. Sci. Monit. 10: CR143-CR151), and/or duplications (Soler, A., et al., Prenat. Diagn. 2003. 23: 319-322).

Preferably, the chromosomal imbalance is associated with or characteristic of Down syndrome, Turner syndrome, Edwards' syndrome, Patau's syndrome, Di-George syndrome, Williams syndrome (WS) and Duchenne muscular dystrophy (DMD), Miller-Dieker, Smith-Magenis, Neurofibromatosis and Steroid sulfatase deficiency. For additional chromosomal imbalances which can be identified using the method of the present invention see Table 1 in Bejjani B., et al., 2005, Am. J. Med. Genet. 134A: 259-267, which is fully incorporated herein by reference.

Preferably, the chromosomal imbalance is an imprinting-associated chromosomal imbalance. As used herein the phrase “imprinting-associated chromosomal imbalance” refers to an abnormal imprinting pattern of a gene or a locus which is either inherited from a parental chromosome or occurred de novo, usually during gametogenesis. For example, a gene which is usually active when inherited from the maternal chromosome (e.g., the UBE3A gene associated with Angelman syndrome) and is silent (i.e., inactive, non-transcribed) when inherited from the paternal chromosome can be subject to an “imprinting mutation”, i.e., an abnormal imprinting pattern which results in silencing of the gene inherited from the paternal chromosome, thus resulting to an individual with both alleles being silent. Non-limiting examples of pathologies caused by aberrant inheritance of imprinting include Prader-Willi syndrome (PWS), Angelman syndrome (AS), Beckwith-Wiedemann syndrome (BWS) and/or pathologies associated with abnormal non-random X inactivation which results in the expression of a mutated recessive gene (e.g., DMD in a heterozygote female).

The phrase “individual devoid of the chromosomal imbalance” as used herein refers to any individual mammal as described hereinabove, which exhibits normal karyotype with balanced chromosomes (i.e., no deleterious inversions, translocations, deletions or duplications), is devoid of any known genetic or epigenetic associated disease, syndrome or disorder, including acquired chromosomal rearrangements, duplications or deletions that are associated with cancer.

It will be appreciated that prenatally identifying a chromosomal imbalance in a conceptus can be also effected by determining the interallelic epigenetic pattern in a cell of the conceptus (see Example 3 of the Examples section which follows). The type of cell of the conceptus used depends on the method used to retrieve such a cell. In case of an ongoing pregnancy, a cell such as a blood cell, an amniotic cell, an extraembryonic membrane cell and/or a trophoblast cell can be used. Such cells can be retrieved from cord blood (using e.g., cordocenthesis), amniotic fluid (using amniocentesis), chorionic villus sampling (CVS), placenta trophoblasts shed into the uterine and/or the cervix (using aspiration, cytobrush, cotton wool swab, endocervical lavage and intrauterine lavage) and fetal cells recovered using foetoscopy. It will be appreciated that in case of a terminated pregnancy (with a fallen, aborted or dead conceptus) any cell of the conceptus can be used.

Preferably, the cell of the conceptus is derived from amniotic fluid, CVS, cord blood and the placenta.

According to the method of this aspect of the present invention the interallelic epigenetic pattern of the cell of the conceptus is compared to that of an unaffected individual devoid of the chromosomal imbalance.

Additionally or alternatively, the interallelic epigenetic pattern as determined in the cell of the conceptus or the maternal cell of the pregnant female carrying the conceptus can be compared to the interallelic epigenetic pattern of the same cell(s) following culturing in the presence or absence of the epigenetic modifier agent as described hereinabove.

Thus, the present invention provides a prenatal diagnostic test based on small aliquots of cells derived from either the pregnant female (e.g., maternal cells from the peripheral blood) or the conceptus (e.g., derived from amniotic fluid or CVS) that can identify a conceptus with various abnormalities such as trisomies, monosomies, micro deletions, small duplications and various genetic defects. The test offers a decisive advantage for detection of fetal abnormalities. When using maternal cells the test offers a non-invasive, non-hazardous method for early detection of fetal abnormalities. The diagnostic test is based on comparing the methylation pattern or chromatin conformation pattern of selected loci in maternal cells (e.g., lymphocytes) pretreated with an epigenetic modifier agent (e.g., DNA-methylation/demethylation inhibitor, histone methylation/demethylation inhibitor or histone acetylation/deacetylation inhibitor) with that of cultured maternal cells untreated with the epigenetic modifier agent as well as to uncultured maternal cells (e.g., as derived from the biological sample, e.g., blood), viewed by molecular means such as MSAP. Alternatively, the methylation pattern or chromatin conformation pattern of selected loci in the maternal cells can be compared to cells of an unaffected individual (cultured in the presence or absence of the epigenetic modifier agent or being uncultured). Still alternatively, the methylation pattern or chromatin conformation pattern of selected loci in cells of the conceptus (uncultured or cultured in the presence or absence of the epigenetic modifier agent) can be compared to that of cells of an unaffected individual (uncultured or cultured in the presence or absence of the epigenetic modifier agent) as well as to conceptus cells which are treated or untreated with the epigenetic modifier agent.

As mentioned above, chromosomal imbalances can be associated with alterations in the interallelic replication timing. For example, as is shown in FIGS. 1a-b, 2a-c, 3a-c and 4a-c, and is described in Example 2 of the Examples section which follows, lymphocytes derived from patients with microdeletions such as Di-George syndrome, VCFS/DIGS and Williams syndrome exhibited alterations in the interallelic replication pattern as compared to lymphocytes of individuals devoid of chromosomal imbalances.

While further reducing the present invention to practice and as is shown in Example 4 of the Examples section which follows, the present inventor has uncovered that chromosomal imbalances in a conceptus can be identified prenatally by detecting the interallelic replication pattern in a cell of the conceptus and/or in a maternal cell derived from the pregnant female carrying the conceptus.

The phrase “interallelic replication pattern” refers to the mode of replication of each of the alleles in a certain locus in a cell. As described in the background section-loci and/or genes which are transcriptionally active in a specific cell replicate early, and loci and/or genes which are transcriptionally inactive replicate late. The replication state of each allele can be detected using the FISH replication assay as described in Example 2 of the Examples section which follows and in PCT, WO 02/023187 A2 and U.S. Pat. No. 6,803,195 B1 to the present inventor, which are fully incorporated herein by reference. In this assay, the synchrony between the replication pattern of both alleles is determined in cells in the S phase. Cells in which both alleles replicate early exhibit two doublet signals (DD), and cells in which both alleles replicate late exhibit two singlet signals (SS). On the other hand, cells in which one allele replicate early and the other allele replicate late exhibit one double signal and one singlet signal (SD). The appearance of SD cells is an indication for asynchrony.

Thus, the present invention contemplates a prenatal diagnostic test which is based on determining the interallelic replication pattern in cells of the conceptus or the maternal cells of the pregnant female carrying the conceptus and comparing such an interallelic replication pattern to cells of an unaffected individual which is devoid of the chromosomal imbalance. Additionally or alternatively, the interallelic replication pattern can be compared between cells of the conceptus or the maternal cells as described hereinabove which are cultured in the presence or absence of an epigenetic modifier agent. Alterations in the interallelic replication pattern between the treated and untreated cells indicate the presence of a conceptus with imbalanced chromosome(s). In addition, the interallelic replication pattern can be compared between cells of the conceptus or the maternal cells as described hereinabove which are cultured as described hereinabove or which are uncultured (e.g., as derived from maternal or fetal blood, or fetal amniocytes).

Chromosomal imbalance mosaicism is a condition in which a chromosomal imbalance exists in only a portion of the cells of the individual. Thus, an individual with chromosomal imbalance mosaicism exhibits two or more cell populations of different chromosomal constitutions [i.e., with balanced (normal) chromosomes and imbalanced chromosomes], all of which derived from a single zygote.

The level of mosaicism, i.e., the percentage of cells having the imbalanced chromosome(s), may affect the severity of the phenotype resulting from or associated with the chromosomal imbalance. In addition, mosaicism can be tissue specific. For example, in a certain tissue (e.g., blood) all cells can be normal, i.e., devoid of the chromosomal imbalance, and in another tissue (e.g., brain) a significant portion of cells can exhibit imbalanced chromosome(s).

The currently practice methods of detecting chromosomal imbalance mosaicism in an individual in need thereof (e.g., an individual with a suspected genetic disease, disorder or syndrome) rely on subjecting cells of the individual to genetic testing (e.g., karyotype and/or FISH analyses). In order to detect tissue-specific mosaicism, such cells are usually derived from at least two types of cells/tissues, such as blood, bone marrow or skin. In addition, in case of low percentage of cells with chromosomal imbalance, the sensitivity (i.e., detection level) of such analyses is limited.

While further reducing the present invention to practice, it was uncovered by the present inventor that a chromosomal imbalance mosaicism can also result in interallelic epigenetic alterations and/or interallelic replication pattern alterations which can be detected in either cells having the chromosomal imbalance or cells devoid of the chromosomal imbalance. In addition, such alterations can be detected prenatally or at any time after birth, e.g., in a child or an adult.

In contrast to currently practiced approaches, the method according to this aspect of the present invention enables the detection of chromosomal imbalance mosaicism with high sensitivity and preferably by using only one type of cells (e.g., blood). Thus, the method according to this aspect of the present invention detects chromosomal imbalance mosaicism by the interallelic epigenetic alterations associated with such chromosomal imbalances in cells of the individual. Since the chromosomal imbalance induced by one type of cells can be detected by the epigenetic alteration caused in another type of cell, the method of the present invention enables the detection of mosaicism even in cells of the individual which are devoid of the chromosomal imbalance.

It should be noted that the chromosomal imbalance mosaicism can be also identified by detecting the interallelic replication pattern in cells of the individual, using, for example, the FISH replication assay. As described hereinabove, such a cell can be a normal cells devoid of the chromosomal imbalance or can be a cell with the chromosomal imbalance.

Non-limiting examples of imbalanced chromosomal mosaicism which can be detected by the method of this aspect of the present invention include Down syndrome mosaicism, fragile X mosaicism, trisomy 9 mosaicism, trisomy 18 mosaicism, trisomy 16 mosaicism, trisomy 15 mosaicism, trisomy 20 mosaicism and 22q11.2 deletion mosaicism.

It will be appreciated that in cases of a small percentage of cells with imbalanced chromosome(s) (i.e., low mosaicism, say less than 30%, less than 20%, or less than 15%), the interallelic epigenetic pattern alterations and/or the interallelic replication pattern alterations may be subtle, thus requiring the use of a plurality of loci and/or detection methods, including an automatic device for analyzing multiple loci.

As is mentioned hereinabove, the present invention contemplates the use of a plurality of loci and/or a combination of methods for detecting interallelic epigenetic patterns (which detect DNA methylation patterns and/or chromatin conformation pattern) and/or interallelic replication patterns, which provide comprehensive analysis for both cancer diagnosis, prenatal diagnosis and/or diagnosis of chromosomal imbalance mosaicism. It will be appreciated that various devices can be used to analyze the data obtained by such methods. These include the TaqMan™ and/or the LightCycler™ systems for the interallelic epigenetic patterns and various microscope systems which enable identification and storage of cell coordinates and signals such as the BioView Duet™ (Bio View Ltd, Rehovot, Israel) and the Applied Imaging System (Newcastle, England) for the interallelic replication patterns.

The agents of the present invention which are described hereinabove for detecting the interallelic epigenetic patterns and/or the interallelic replication pattern may be included in a diagnostic kit/article of manufacture preferably along with appropriate instructions for use and labels indicating FDA approval for use in diagnosing cancer and/or prenatal diagnosis of a conceptus with imbalanced chromosome(s) using maternal and/or conceptus cells.

Such a kit can include, for example, at least one container including at least one of the above described diagnostic agents (e.g., probes for monoallelically expressed loci such as SNRPN, methylation sensitive restriction enzymes such as HpaII-MspI, FISH probes) and an imaging reagent packed in another container (e.g., labeled secondary antibodies, buffers, chromogenic substrates, fluorogenic material). The kit may also include appropriate buffers and preservatives for improving the shelf-life of the kit.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological, recombinant DNA techniques and cytogenetics. Such techniques are thoroughly explained in the literature. See, for example, “The principles of Clinical Cytogenetics” Gersen S. L. and Keagle M. B., Eds. (1999); “Molecular Cloning: A laboratory Manual” Sambrook et al., (1989); “Current Protocols in Molecular Biology” Volumes I-111 Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### DNA and Chromatin-Based Methods for Detecting Changes in Interallelic Epigenetic Patterns

The present invention relates to methods of detecting cancer and cancer risk.

The underlying model: Cancer cells may induce monoallelic epigenetic changes which can be detected in non-cancerous cells—The present inventor has hypothesized that cancer cells excrete some chemical compounds which may affect gene non-specific epigenetic changes in cells of the individual, not necessarily the tumor cells but also e.g., blood cells (such as lymphocytes) in cases of a solid tumor. Such chemicals induce detectable changes in epigenetic patterns of the cancer-stricken individuals such as interallelic changes in DNA methylation and/or chromatin conformation of loci that are not associated directly with the specific cancer (e.g., SNRPN and/or GABRB3, which are associated with PWS-AS in case of prostate cancer and/or renal cell carcinoma). In addition, such interallelic changes can be reversed by treating the cells (during cell replication cycles) with inhibitors of DNA-methylation and/or histone-deacetylation. Thus, it is possible to detect interallelic epigenetic modifications by comparing changes in the pattern of DNA methylation and/or chromatin conformation in non-induced cells (e.g., lymphocytes isolated from a blood sample) as well as in mitotic induced cells (e.g., PHA-stimulated lymphocytes). To confirm that the changes are due to cancer, the cells can be further grown in the presence or absence of methylation or acetylation inhibitors. In this analysis it is expected that in healthy individuals there will be minor differences in the pattern of treated and untreated lymphocytes while in cancer-stricken individuals there will be remarkable differences between the treated and untreated samples. The analysis can be carried out on both biallelically expressed genes or loci, monoallelically expressed genes or loci or non-coding loci.

The method requires analysis of the pattern of DNA methylation by RFLP's technology and of chromatin deacetylation and/or methylation by digesting the chromatin with specific enzymes such as micrococcal nuclease and running the digested chromatin on a gel; a change in conformation is expressed in differential band migration. These epigenetic modifications associate with genetic functioning within cells of an animal, including a human animal.

The method steps—The invention appraising the prognosis and/or risk of cancer comprises the following steps:

(a) Cells including non-malignant or malignant cells are obtained from an individual. Preferably, the cells are derived from a body tissue or body fluid. The body tissue is preferably bone marrow. The body fluid is preferably selected from blood, amniotic fluid, urine, and saliva. Preferably, the blood is peripheral blood. The cells are preferably lymphocytes; It should be noted that unlike the current practice which concentrate on diagnosing cancer in the cancerous cells (e.g., leukemia in bone marrow, breast cancer in a breast tissue biopsy), the cells used according to the method of the present invention can be any cells of the individual which are subject to interallelic epigenetic modifications due to the cancer.

b) The lymphocytes are preferably subjected to a growth stimulus before step (c), preferably using phytohemagglutinin (PHA);

(c) In case blood cells are obtained from the individual they are prepared for short-term culturing in F10 medium supplemented with 20% fetal calf serum (FCS), 3% phytohemagglutinin (PHA), 0.2% heparin, and 1% antibiotics (a standard solution of penicillin and streptomycin). To verify that the cells underwent cell division, following incubation at 37° C. for 72 hours the cells were subjected to colchicine treatment at a final concentration of 0.1 μg/ml for one hour and then incubated with hypotonic buffer (0.075 M KCl at 37° C. for 15 minutes) and washed 4 times, each with a fresh cold 3:1 methanol:acetic acid solution.

In order to compare the effect of the epigenetic modulating agents on the epigenetic state, a portion of the cells is subjected to inhibitors of DNA methylation or histone modifications associated with gene expression and/or chromatin remodeling. Briefly, cells are cultured in the presence of 10⁻⁷M 5-azacytidine (AZA; Sigma Chemical, St. Louis, Mo. USA), a methylation blocking agent (Haaf T., 1995, The effects of 5-azacytidine and 5-azadeoxycytidine on chromosome structure and function; implications for methylation-associated cellular processes. Pharmac Ther 65:19-46), added to the other ingredients of the medium, and zebularine, a stable DNA cytosine methylation inhibitor, that is preferentially incorporated into DNA, and in addition, preferentially depletes DNA methyltransferase 1 (DNMT1) (Cheng J C., et al., 2003, Inhibition of DNA methylation and reactivation of silenced genes by zebularine. I Natl. Cancer Inst 95:399-409).

(d) Lymphocyte nuclei are isolated in a Hamilton buffer essentially as described (Van Blokland R., et al., 1997, Condensation of chromatin in transcriptional regions of an inactivated plant transgene: evidence for an active role of transcription in gene silencing. Mol Gen Genet. 257:1-13; Saxena P, et al., 1985, An efficient procedure for isolation of nuclei from plant protoplasts. Protoplasma 128:184-189), washed twice with FACS buffer [10 mM MES (2-Morpholinoethanesulfonic acid)], 0.2 M sucrose, 0.01% Triton X-100, 2.5 mM EDTA, 2.5 mM dithiothreitol [Saxena, 1985 (Supra)] to remove soluble contaminants, and passed through two layers each of 150- and 100-μm filters to remove cell debris. In order to isolate live cells, the nuclei can then be subject to FACS analysis by precipitation (1000 g, 7 minutes, 4° C.), and resuspension in FACS buffer supplemented with 50 μg/ml DNase-free RNase A (Roche Molecular Biochemicals) and 50 μg/ml Propidium Iodide (PI, Sigma, which incorporates only to live cells), using the FACSort (Becton Dickinson). The position of PI fluorescence intensity for G0/G1 nuclei has been changed from one experiment to another as a result of alteration in the amplifier gains for FL-2, which was necessary to accommodate fluorescence intensity of both G0/G1 and G2 nuclei. PI-positive cells are sorted by the FACS analysis and are kept for further analysis.

(e) Optionally, prior to FACS analysis the nuclei can be pulse-labeled with BrdUrd in order to isolate dividing cells in the S phase. Briefly, nuclei reactivated for S phase (72 hour after their preparation) are pulse-labeled for 30 minutes with 10 μM BrdUrd (Sigma), after which nuclei are isolated, stained with Propidium Iodide (PI; 50 μg/ml PI, Sigma) and analyzed by FACS analysis using the FACSort (Becton Dickinson). BrdUrd-positive/PI-positive cells are sorted by the FACS analysis and are kept for further analysis.

(f) DNA is prepared from the FACS sorted or unsorted cell nuclei (from about 50,000 nuclei) and is kept for further DNA or chromatin-based epigenetic analysis. Optionally, to confirm that the cell nuclei are derived from dividing cells at the S phase, a portion of the DNA is resolved on 0.8% agarose gel, transferred onto nitrocellulose membrane, and probed with anti-BrdUrd antibody (Becton Dickinson).

(g) Methylation-based epigenetic analysis is performed on the DNA using specific markers, i.e., probes of genes/loci known to exhibit interallelic epigenetic changes due to cancer.

One option is to test genes that are expressed biallelically. The locus or loci can be selected from tumor-associated genes and non-coding loci associated with chromosomal segregation. The tumor-associated genes are preferably selected from oncogenes, tumor suppressor genes, and transcription factors involved in translocations associated with blood tumors. For example, the locus or loci of the biallelically expressed genes are HER2, CMYC, TP53, RB1, TP53, AML1,

Another option is to test genes from locus or loci that are expressed monoallelically. The monoallelically expressed locus or loci are preferably selected from imprinted loci, loci where one allele has been silenced, and loci on the X-chromosome in female individuals. Preferably, the loci are selected from the group of tumor-associated genes, satellite DNA and imprinted loci. The tumor-associated genes are preferably selected from oncogenes, tumor suppressor genes, and transcription factors. The imprinted locus is preferably selected from the Prader-Willi syndrome locus. For example, the locus or loci of the monoallelically expressed genes are 15q11-13 (which includes e.g., SNRPN and GABRB3) and 11p15.

Another option is to use locus or loci that are non-coding loci and lack transcriptional capability. The non-coding locus or loci are preferably selected from DNA sequences associated with chromosome segregation. The DNA is preferably satellite DNA. For example, the locus or loci that are non-coding are centromeric repetitive arrays such as alpha II and III satellites for all chromosomes, preferably CEN17, CEN15, CEN11 and CEN10.

(h) Indication of cancer—DNA methylation pattern or chromatin conformation pattern is compared in the above genes/loci in lymphocytes of unaffected individuals and in individuals with a suspected cancer. In addition, in cells of either cancer suspected or unaffected individuals the DNA methylation or chromatin conformation patterns are compared between cell cultures which are treated with an epigenetic modifying agent (e.g., methylation-inhibitors or chromatin condensations modifiers) and untreated cultures. In cancer suspected individuals the DNA methylation or chromatin conformation patterns are different between cell cultures treated with epigenetic modifying agents and untreated cell cultures. On the other hand, in the unaffected individuals, the DNA methylation or chromatin conformation patterns are not significantly affected by the presence of the epigenetic modifying agent in the cell culture.

Thus, according to the method of the present invention, a reversible alteration (due to treatment with an epigenetic modifying agent) in the methylation pattern or chromatin conformation pattern in cells of an individual, is indicative of cancer or cancer risk. That change can be associated, for example, with a methylation of one allele in a biallelically expressed locus, which is normally unmethylated on both alleles, or with a demethylation of one allele in a non-coding locus, which is normally methylated on both alleles. On the other hand, the change can be associated with a methylation of a second allele in a monoallelically expressed loci (which is normally methylated on one allele and unmethylated on the other allele) or with a demethylation of a second allele in a monoallelically expressed loci.

Criteria for selecting loci for analysis—The locus which is used for cancer diagnosis is selected from a monoallelically expressed locus, a biallelically expressed locus and/or a non-coding locus. Since the cancer induces epigenetic changes not necessarily related to the chromosomal imbalance or the aberrant gene regulation associated with the cancer, the locus which is used for detection of interallelic epigenetic modifications can be unrelated to the specific cause of cancer in question, e.g., can be on another chromosome.

For example, detection of breast cancer, which is usually related to aberrations (e.g., duplication, overexpression, null mutations) in genes such as HER2, BRCA1, BRCA2, TP53 can be tested using the SNRPN locus, which is associated with the PWS-AS on chromosome 15q11-13. Thus, the methylation pattern in SNRPN in a normal, unaffected individual is such that one allele is methylated and the other allele is unmethylated. In cells (e.g., lymphocytes) of an individual with breast cancer or prostate cancer, an alteration in the methylation pattern of SNRPN would be that either both alleles are methylated on the SNRPN locus or both alleles are unmethylated. Since the cancer induces random epigenetic changes, which may affect only one allele, such interallelic changes, which are reversible in the presence of the epigenetic modifying agents [which are capable of modifying methylation pattern in pathologies but not the normal, inherited methylation patterns (Egger G. et. al., 2004)] can be indicative for the presence of cancer.

The following epigenetic modifying agents can be used according to the method of the present invention.

DNA methylation inhibitors—5-azacytidine (AZA; 5-aza-CR), 5-aza-2′deoxycytidine (5-aza-CdR), 5 fluorocytosine, pseudoisocytosine, Zebularine, and Procainamide, but not limited to other chemicals that inhibit DNA methylation.

Chromatin modifying agents—Histone deacetylase inhibitors include Trichostatin A (TSA), Sodium butyrate and N-nitroso-n-methylurea, but not limited to other histone acetylated agents.

Methods of detecting DNA methylation—A number of methods of the art of molecular biology are not detailed herein, as they are well known to the person of skill in the art. Such methods include PCR cloning, Southern hybridization, restriction fragment length polymorphism (RFLP) analysis, amplified fragment length polymorphism (AFLP) analysis, methylation-sensitive amplification polymorphism (MSAP) analysis, scoring of AFLP and MSAP bands, sequence analysis, analysis of chromatin conformation, transformation of bacterial and yeast cells, transfection of mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, Short Protocols in Molecular Biology, by F. M. Ausubel et al. (eds.) 3rd ed. John Wiley & Sons; ISBN: 0471137812, 1995, and A Manual of Online Molecular Biology Techniques by Ed Rybicki, Dept. of Molecular and Cell Biology, University of Cape Town, South Africa, 2005. These publications are incorporated herein in their entirety by reference. In particular, the isolation of cells from the body of an animal, and the analysis thereof by fluorescent absorption of whole nuclei or the pattern of DNA methylated in specific genes, have been described in several articles and textbooks, see e.g. the publication of Cedar H, et al., 1979, Direct detection of methylated cytosine in DNA by use of the restriction enzyme Mspl. Nucleic Acids Res 6:2125-2132; Eden S., et al., 1998, DNA methylation models histone acetylation. Nature 394:842; Hashimshony T., et al., 2003, The role of DNA methylation in setting up chromatin structure during development. Nature Genet. 34: 187-192; Doerksen T., et al., 2000, Deoxyribonucleic acid hypomethylation of male germ cells by mitotic and meiotic exposure to 5-azacytidine is associated with altered testicular histology. Endocrinology 141: 3235-3244; Watson R E, Goodman J I. 2002, Epigenetics and DNA methylation come of age in toxicology. Toxicol Sci 67:11-16; Lorincz M C, et al., 2004, Intragenic DNA methylation alters chromatin structure and elongation efficiency in mammalian cells. Nature Structural and Molecular Biology 11:1068-1075; Lund G, et al., 2004, DNA methylation polymorphisms precede any histological sign of atherosclerosis in mice lacking apolipoprotein E J Biol. Chem 279:29147-29154; included in their entirety by reference.

Decondensation Assay—Equal amounts of nuclei (determined by pack volume and relative density) prepared from cells of unaffected individuals (e.g., healthy and not predisposed to cancer) or cancer affected individuals (including cancer suspected individuals, cancer affected individuals and cancer predisposed individuals) are first permeabilized by incubation in Hamilton buffer containing 0.15% Triton X-100, washed and resuspended in 600 μl of nuclei digestion buffer (50 mM Tris-HCl, pH 8.0, 0.3 M sucrose, 5 mM MgCl₂, 1.5 mM NaCl, 0.1 mM CaCl₂, and 5 mM-mercaptoethanol, essentially as described in Van Blokland R, et al., 1997 (Condensation of chromatin in transcriptional regions of an inactivated plant transgene: evidence for an active role of transcription in gene silencing. Mol Gen Genet 257:1-13). A sample (80 μl) is removed for untreated control. MNase (micrococcal nuclease) (1000 units/ml) is added and at various time points, samples (80 μl) are taken, mixed with 350 μl of stop solution (2 mg/ml proteinase K (Roche Molecular Biochemicals), 10 mM NaCl, 1 mM MgCl₂, 10 mM Tris-HCl, pH 7.5, and 2% SDS, and incubated overnight at 37° C. To prepare DNA, 140 μl of 5M potassium acetate are added to each sample, mixed well, incubated on ice for 15 minutes, and centrifuged (15 minutes, 12,000 g, 4° C.). The supernatant is collected, extracted once with phenol/chloroform/isoamyl alcohol (25:24:1), and DNA is precipitated by adding 1 ml of 100% ethanol, followed by centrifugation. The DNA pellet is resuspended in 30 μl of TE (10 mM Tris-HCl, pH 8.0, 1 mM EDTA), treated with RNase A (20 μg/ml, 25 minutes at room temperature), and nuclease digestion products are resolved on 1.6% agarose gels and stained with ethidium bromide.

Criteria for selecting a probe from a specific locus—For molecular studies the size of the probe depends on the sensitivity of the method and it should be locus specific and preferably, with low percentage of polymorphism (to avoid complex signals and discriminating between disease-dependent alteration and simple polymorphism between individuals).

### Example 2

### Identification of Chromosomal Imbalances Using the Fish Replication Assay

The presence of chromosomal imbalances such as DiGeorge syndrome (associated with a deletion on chromosome 22q11.2), Velo-Cardio-Facial syndrome/DiGeorge syndrome (VCFS/DIGS) (associated with a deletion on chromosome 22q11.2) and Williams syndrome (WS) (associated with a deletion on chromosome 7q11.23) was identified by comparing the interallelic replication patterns of the affected individuals with those of healthy, unaffected individuals, as follows.

Probes used for the FISH replication assay—The SNRPN probe, derived from the PWS-AS critical region on chromosome 15q11-13 was obtained from Vysis 32-190004); the RB1 probe, derived from chromosome 13q24 was obtained from Vysis 32-190001); the ARSA probe, derived from the long arm of chromosome 22, distal to 22q11.2 was obtained from Vysis 32-191028).

Identification of DiGeorge syndrome using the FISH replication assay and the SNRPN probe—The percentage of cells in which one allele of the SNRPN gene replicates early and the other allele replicates late (i.e., SD cells) was determined in lymphocytes of control subjects or of subjects affected with Di-George syndrome and which exhibit the characteristic deletion on chromosome 22q11.2. As is shown in FIGS. 1a-b, while in control samples the percentage of cells having asynchronous replication was high (SD values in the range of 44-57%) as expected from the SNRPN gene (which is monoallelically expressed), the percentage of cells having asynchronous replication in the Di-George syndrome group of patients was significantly low (SD values in the range of 23-33%), which is similar to what expected from a biallelically expressed gene and not from a monoallelically expressed gene.

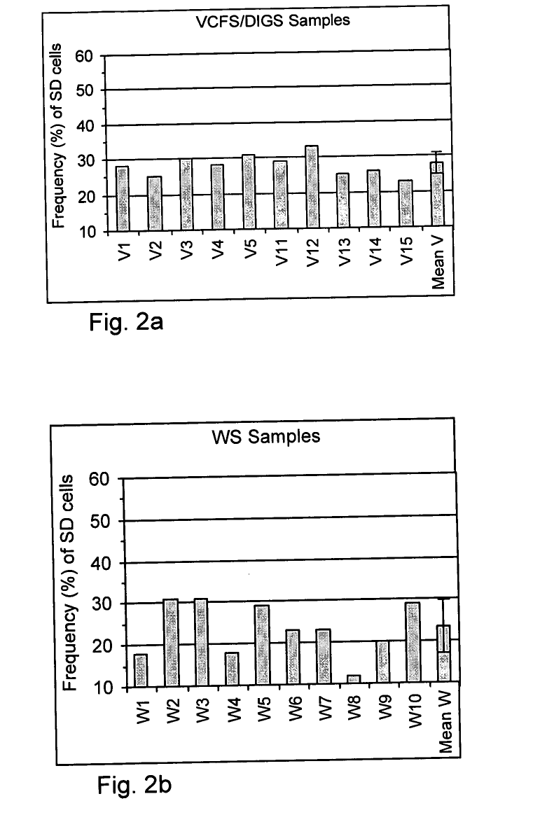

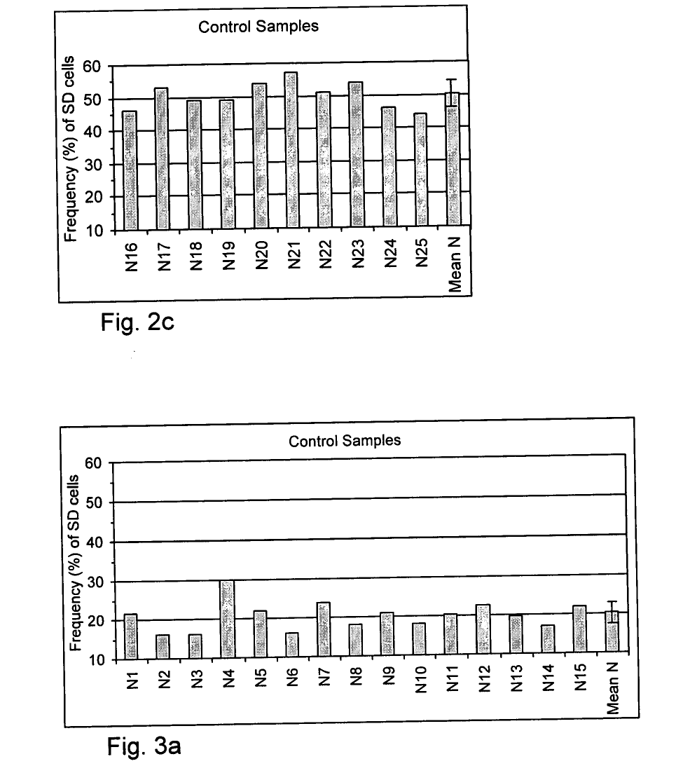

Identification of Velo-Cardio-Facial syndrome/DiGeorge syndrome and Williams syndrome using the FISH replication assay and the SNRPN probe—As is shown in FIGS. 2a-c, while the control subjects exhibited high SD values (in the range of 42-57%), characteristic of an imprinted locus, the two groups of patients show similar and significantly low SD values (in the range of 22-32% for VCFS/DIGS and 11-31% for WS), not characteristic of an imprinted locus such as SNRPN.

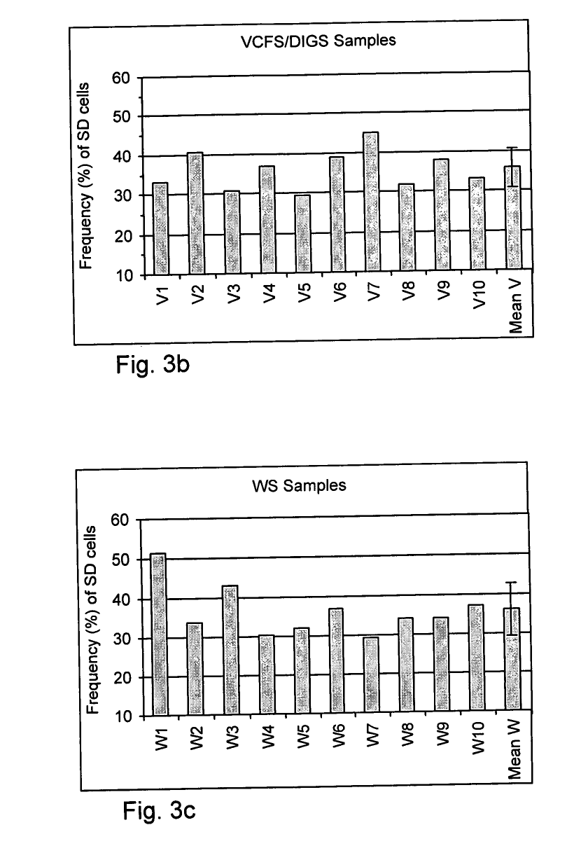

Identification of Velo-Cardio-Facial syndrome/DiGeorge syndrome and Williams syndrome using the FISH replication assay and the RBI probe—As is shown in FIGS. 3a-c, while the control subjects exhibited low SD values (in the range of 15-30%), characteristic of a biallelically expressed locus, the two groups of patients show similar and significantly high SD values (in the range of 29-45% for VCFS/DIGS and 29-51% for WS), not characteristic of biallelically expressed locus such as RB1.

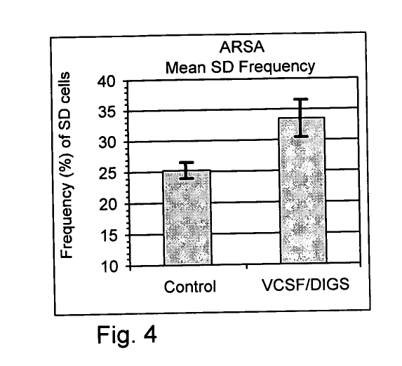

Identification of Velo-Cardio-Facial syndrome/DiGeorge syndrome using the FISH replication assay and the ARSA probe—As is shown in FIG. 4, while in the control subjects the percentage of cells with an asynchronous replication of the ARSA locus (which is derived from the long arm of chromosome 22, distal to 22q11.2) was low with an average SD value of 25%, characteristic of a biallelically expressed locus, the percentage of cells with an asynchronous replication of the ARSA locus in the VCSF/DIGS subjects was significantly higher, with an SD value of 33% (P<0.0005; Student's t-test), as expected from a monoallelically expressed locus and not from a biallelically expressed locus such as ARSA.

Altogether, these results demonstrate the identification of chromosomal imbalances associated with various genetic syndromes by the detection alterations in the interallelic replication pattern in cells of the affected individuals.

### Example 3

### Prenatal Diagnosis by Determining Interallelic Epigenetic Pattern Modifications

Current prenatal diagnosis is performed on fetal cells obtained from amniotic fluid and/or CVS samples in which genetic disorders resulting from chromosomal aberrations or DNA point mutations are detected using cytogenetic or molecular analyses. However, in many cases, small deletions and duplications or imbalanced rearrangements (e.g., de novo formed translocations or inversion), remain undetectable using the common cytogenetic analyses. In addition, epigenetic errors—that is, errors involving information other than changes in DNA sequences, usually established in parental germ cells and inherited post fertilization to the offspring, are usually neglected.

The working hypothesis underlying the method of prenatal diagnosis according to the present invention—A fetus with an imbalanced chromosome(s) may excrete some chemical compounds to maternal blood cells, preferably peripheral blood lymphocytes, that modify the epigenetic pattern of at least one allele at a certain locus, e.g., affect the pattern of DNA methylation in specific coding and non coding DNA sequences and/or of chromatin conformation in definite chromosomal regions. The modifications in the epigenetic pattern can be detected in non-cultured lymphocyte cells as well as in PHA-stimulated lymphocytes grown in the presence or absence of DNA-methylation inhibitors or histone-deacetylation inhibitors and be compared to those of cells of unaffected individuals. In this analysis it is expected that in mothers having healthy fetuses there will be minor differences in the pattern of treated and untreated lymphocytes while in mothers having fetuses with chromosomal abnormalities and genetic defects (imbalanced chromosomes) there will be remarkable differences between the treated and untreated samples. In addition, there will be a remarkable difference in the epigenetic pattern of uncultured maternal cells derived from females carrying normal fetuses and females carrying fetuses with imbalanced chromosomes. The analysis is carried out on genes and/or loci which are biallelically-expressed, monoallelically expressed and/or non-coding as is further described hereinbelow.

The present invention offers the use of epigenetic profile for prenatal diagnosis on two levels: (i) applied to fetal cells, obtained either through amniocentesis, chorionic villus sampling (CVS) or fetal blood sampling; and (ii) applied to maternal peripheral blood cells, and thus offering a non invasive and non risky method, enabling to achieve information on the genetic makeup of the fetus.

Consequently, this invention relates to methods for the detection of prenatal chromosomal and genetic disorders in a conceptus (e.g., an embryo or a fetus, including aborted fetuses or stillborns) using an invasive or a non-invasive method. The methods require analysis of DNA methylation and/or of chromatin deacetylation and/or methylation in various loci within cells of an animal, including human.

The invention is based on the following:

(1) Molecular determination of changes in DNA methylation and/or chromatin conformation can be done either in cultured or uncultured fetal cells or maternal blood cells (e.g., lymphocytes);

(2) Determination is done by comparing the DNA methylation and/or chromatin conformation in cells of the conceptus or the maternal cells carrying the conceptus and an unaffected individual, and in cells of the same individual uncultured, untreated during culturing and treated during culturing with methylation or deacetylation inhibitors;

The practice of the invention involves methods known in the art of molecular biology as described in Example 1, hereinabove.

Molecular Analysis (Epigenetic Analysis)

(a) Obtaining maternal cells (e.g., from blood, lymphocytes) or fetal cells (e.g., amniocytes or CVS) and optionally culturing the cells.

The cells are preferably subjected to a growth stimulus preferably using phytohemagglutinin (PHA). Preferably, during culturing, the cells are also subjected to methylation inhibitors or histone modifiers associated with gene expression and/or chromatin remodeling;

Culturing of blood cells (fetal cells obtained from cord blood or maternal cells obtained e.g., from peripheral blood) (e.g., for short term culturing) is performed in F10 medium supplemented with 20% fetal calf serum (FCS), 3% phytohemagglutinin (PHA), 0.2% heparin, and 1% antibiotics (a standard solution of penicillin and streptomycin). Cultures are incubated at 37° C. for 72 hours, optionally colchicine (final concentration 0.1 μg/ml) is added for 1 hour, followed by hypotonic treatment (0.075 M KCl at 37° C. for 15 minutes) and four washes, each with a fresh cold 3:1 methanol:acetic acid solution. Corresponding blood samples, taken from the same individuals, are also cultured in the presence of 10⁻⁷ M 5-azacytidine (AZA; Sigma Chemical, St. Louis, Mo. USA), a methylation blocking agent (Haaf 1995), added to the other ingredients of the medium, and zebularine, a stable DNA cytosine methylation inhibitor, that is preferentially incorporated into DNA, and in addition, preferentially depletes DNA methyltransferase 1 (DNMT1) (Cheng et al., 2003).

Alternatively, cell samples derived from amniotic fluid or CVS are cultured in the presence of a mixture of CHANK and F-10 media (pH 7.0-7.5) at 37° C. and described hereinabove for blood samples.

(b) Preparing DNA or chromatin (histone+DNA) from cells cultured as described hereinabove or uncultured cells, as described in Example 1, hereinabove.

(c) Determining the methylation pattern of specific genes/loci including biallelically-expressed loci, monoallelically-expressed loci and non-coding loci, as follows.

Monoallelically expressed genes and/or loci—The monoallelically-expressed genes and/or loci are preferably selected from imprinted loci, loci where one allele has been silenced, and loci on the X-chromosome in female individuals. Preferably, the loci are selected from imprinted loci such as GABRB3, the Prader-Willi syndrome locus (e.g., SNRPN) and D15S10. Loci on the X-chromosome in female individuals which are subject to X-inactivation. XIST is located on the X-chromosome and is responsible for X-inactivation and, as such, is activated only on the inactive X-chromosome. This gene is a classical example of monoallelically expressed genes.

Non-coding loci lacking transcriptional capability—The non-coding loci are preferably selected from DNA sequences associated with chromosome segregation. The DNA is preferably satellite DNA. From centromeric repetitive arrays such as alpha II and III satellites for all chromosomes, preferably CEN17, CEN15, CEN11 and CEN10.

Biallelically-expressed genes and/or loci—preferably HER2, CMYC, TP53, RB1, AML1. Loci on the X chromosome such as STS and KAL which are located within the pseudo-autosomal region of the short arm of the X-chromosome and, as such, are known to escape X-inactivation.

Indication of a Chromosomal Imbalance in a Conceptus

Biallelically expressed loci—In these loci in unaffected individuals both alleles exhibit the same state of methylation (i.e., either both alleles are methylated or both alleles are unmethylated). However, in at least some cells of the conceptus or the maternal cells of the pregnant female carrying the conceptus, in the presence of a chromosomal imbalance (e.g., aneuploid) there is an alteration in the interallelic epigenetic, namely, one allele is methylated and the other allele is unmethylated.

Monoallelically expressed loci—In these loci in unaffected individuals there is a difference in the state of methylation between the two alleles (i.e., one is methylated and the other is unmethylated). However, in case of a chromosomal imbalance there is an alteration in the interallelic epigenetic pattern, namely, either both alleles are methylated or both become unmethylated.

Non-coding loci—In these loci in unaffected individuals both alleles exhibit the same state of methylation (i.e., either both alleles are methylated or both alleles are unmethylated). However, in at least some cells of the conceptus or the maternal cells of the pregnant female carrying the conceptus, in the presence of a chromosomal imbalance (e.g., aneuploid) there is an alteration in the interallelic epigenetic pattern, namely, one allele is methylated and the other allele is unmethylated.

Detection Methods:

The DNA methylation pattern is determined as described in Example 1, hereinabove.

The DNA methylation inhibitors and the chromatin modifying agents are described in Example 1, hereinabove.

Decondensation Assay—as described in Example 1, hereinabove.

### Example 4

### Prenatal Diagnosis by Determining Interallelic Replication Pattern Modifications

As is shown in Example 2, hereinabove, the present inventor has uncovered that loss of small chromosomal segments within a genome can affect the replication pattern of alleles in loci not directly associated with the chromosomal aberrations.

The working hypothesis underlying the method of prenatal diagnosis according to the present invention—A fetus with an imbalanced chromosome(s) excrete some chemical compounds to maternal blood cells, preferably peripheral blood lymphocytes, that modify the pattern of replication in specific coding and non coding DNA sequences and such modification can be compared in cultured or uncultured maternal cells treated or untreated with DNA-methylation inhibitors or histone-deacetylation inhibitors. In this analysis it is expected that in mothers having healthy fetuses there will be minor differences in the pattern of DNA replication of treated and untreated lymphocytes while in mothers having fetuses with chromosomal abnormalities and genetic defects there will be remarkable differences between the treated and untreated cells. In addition, there will be a remarkable difference in the replication pattern of maternal cells derived from females carrying normal fetuses and females carrying fetuses with imbalanced chromosomes. The analysis is carried out on genes and/or loci biallelically-expressed loci, monoallelically expressed loci and/or non-coding loci as described in Example 3, hereinabove.

The present invention offers the use of replication synchrony assay for prenatal diagnosis on two levels: (i) applied to fetal cells, obtained either through amniocentesis, chorionic villus sampling (CVS) or fetal blood sampling; and (ii) applied to maternal peripheral blood cells, and thus offering a non invasive and non risky method, enabling to achieve information on the genetic makeup of the fetus.

The method requires analysis of the pattern of replication of specific genes and/or loci using cytogenetic analysis (e.g., the FISH replication assay) of fetal cells and/or of maternal cells.

FISH Replication Assay

(a) Obtaining fetal cells through amniocentesis, chorionic villus sampling (CVS) or cord blood sampling and optionally culturing them as described in Example 3, hereinabove;

(b) Obtaining maternal cells that are derived from blood (e.g., peripheral blood, lymphocytes) and optionally culturing them as described in Example 3, hereinabove;

(c) The preferred method of determining interallelic replication pattern is FISH. The FISH replication assay is relatively simple and fast, and in contrast to the classical replication timing methods avoids the incorporation of BrdU or other agents that can interfere with DNA replication; selects S-phase cells with no need for cell sorting or cell synchronization; and allows identification of individual alleles within a single cell (Selig et al. 1992; Boggs and Chinault 1997). The FISH assay relies on replication dependent chromatin conformation. Accordingly, the replication status of a locus is inferred from the shape of the hybridization signal obtained at interphase, following FISH with a locus-specific probe. Prior to replication, each identified DNA sequence shows a single dot like hybridization signal (“singlet”; S), while at the end of replication it assumes a doubled bipartite structure (“doublet”; D) (Selig et al. 1992; Mukherjee et al. 1997; Boggs and Chinault 1997). Cells with one “singlet” and one “doublet” represent S-phase cells (designated SD cells) in which only one of the allelic sequences has replicated. Cells with two “singlets” (SS cells) represent those in which both sequences are not yet replicated, and cells with two “doublets” (DD cells) represent those in which both sequences have replicated. In an unsynchronized population of replicating cells the frequency of cells at a given stage expresses the relative duration of that stage. Hence, the frequency of SD cells, out of the total population of cells with two hybridization signals, correlates with the time interval (at S-phase) during which the two allelic counterparts differ in their replication status, i.e., there is an early (identified by a “doublet”) and a late replicating allele (recognized by a “singlet”). Similarly, the frequency of DD cells reveals the relative time interval at interphase during which the two counterparts are replicated (part of S-phase, and the whole G2 phase), while the frequency of SS cells correlates with the time interval during which the two counterparts are unreplicated (G0, G1 and part of S-phase). Thus, a high frequency of SD cells shows asynchrony in replication timing of the two allelic counterparts; high frequency of DD cells indicates early replication of the identified locus; and high frequency of SS cells points to late replication.

The loci used to determine the replication pattern exhibit modification in the replication pattern due to chromosomal and genetic defect in the fetus. These loci be biallelically-expressed loci, monoallelically-expressed loci and non-coding loci as described hereinabove.

Criteria for selecting a probe from a specific locus—a probe for cytogenetic analysis should be large enough for in situ hybridization; about 200 KB and more.

Indication of a Chromosomal Imbalance in a Conceptus

Biallelically expressed loci—In these loci in unaffected individuals both alleles replicate synchronously (i.e., the percentage of SD cells is low). However, in at least some cells of the conceptus or the maternal cells of the pregnant female carrying the conceptus, in the presence of a chromosomal imbalance (e.g., aneuploid) there is an alteration in the interallelic replication pattern (i.e., one replicates early and is shown by FISH as a doublet and one replicates late and is shown by FISH as a singlet) leading to an increase in the percentage of SD cells.

Monoallelically expressed loci—In these loci in unaffected individuals there is a difference in the synchrony of replication (i.e., asynchrony replication patterns, in most cells one allele replicates early and the other replicates late, high percentage of SD cells). However, in case of a chromosomal imbalance there is an alteration in the interallelic replication pattern, namely, the replication pattern of the alleles becomes more synchronized, i.e., the fraction of SD cells decreases as compared to the unaffected individual.

Non-coding loci—In these loci in unaffected individuals both alleles replicate synchronously (i.e., low percentage of SD cells). However, in at least some cells of the conceptus or the maternal cells of the pregnant female carrying the conceptus, in the presence of a chromosomal imbalance (e.g., aneuploid) there is an alteration in the replication pattern, namely, the alleles exhibit an asynchronous replication pattern (i.e., one replicates early and is shown by FISH as a doublet and one replicates late and is shown by FISH as a singlet) leading to an increase in the percentage of SD cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

Amiel A, Litmanovitch T, Gaber, E, Lishner M, Avivi L, Fejgin M. (1997) Asynchronous replication of p53 and 21q22 loci in chronic lymphocytic leukemia. Hum Genet 101:219-222.
Amiel A, Litmanovitch, T Lishner M, Mor A, Gaber E, Fejgin M D, Avivi L. (1998a) Temporal differences in replication timing of homologous loci in malignant cells derived from CML and lymphoma patients. Genes Chromosomes Cancer 22: 225-231.
Amiel A, Avivi L, Gaber E, Fejgin M D. (1998b) Asynchronous replication of allelic loci in Down syndrome. Eur J Hum Genet 6: 359-364.
Amiel A, Korenstein A, Gaber E, Avivi L. (1999) Asynchronous replication of alleles in genomes carrying an extra autosome. Eur J Hum Genet 7: 223-230.
Amiel A, Levi E, Reish O, Sharony R, Fejgin M D (2001) replication status as a possible marker for genomic instability in cells originating from genotype with balanced rearrangements. Chromosome Res 9:611-616.
Amiel A, Reish O, Gaber E, Masterman R, Tohami T, Fejgin M D (2002) Asynchronous replication of alleles in genomes carrying a microdeletion. Isr Med Assoc J 4:702-705.
Avivi L, Dotan A, Ravia Y. (2002) Facile detection of cancer and cancer risk based on level of coordination between alleles. PCT, WO 02/023187 A2.
Avivi L, Dotan A, Ravia Y. (2004) Facile detection of cancer and cancer risk based on level of coordination between alleles. U.S. Pat. No. 6,803,195 B1.
Baylin, S B., Herman, J G. (2000) DNA hypermethylation in tumorigenesis: epigenetics joins genetics. Trends Genet 16:168-174.
Boggs B A, Chinault A C. (1997) Analysis of DNA replication by fluorescence in situ hybridization. Methods 13: 259-270.
Dotan Z A, Dotan A, Litmanovitch T, Ravia, Y, Oniasvili N, Leibovitch I, Ramon J, Avivi L. (2000) Modification in the inherent mode of allelic replication in lymphocytes of patients suffering from renal cell carcinoma: a novel genetic alteration associated with malignancy. Genes Chromosomes Cancer 27:270-277.
Dotan Z A, Dotan A Ramon J, Avivi, L. (2004) Altered mode of allelic replication accompanied by aneuploidy in peripheral blood lymphocytes of prostate cancer patients. Int J Cancer 111: 60-66.
Egger G, Liang G, Aparicio A, Jones P A. (2004). Epigenetics in human disease and prospects for epigenetic therapy. Nature 429:457-463.
Ensminger A W, Chess A. (2004) Coordinated replication timing of monoallelically expressed genes along human autosomes. Hum Mol Genet 13: 651-658.
Frimer H (2000) Peripheral blood lymphocytes of breast cancer patients display loss of synchrony in allelic replication timing associated with aneuploidy. M. Sc. thesis (in Hebrew), Sackler School of Medicine, Tel-Aviv University.
Gimelbrant A, Ensminger A W, Qi P, Zucker J, Chess A. (2005) Monoallelic expression and asynchronous replication of p120 catenin in mouse and human cells. J Biol Chem 280:1354-1359.
Goldshtein A (2004) Replication pattern of alleles in normal cells growing adjacent to aneuploid cells—Research in fibroblasts taken from amniotic fluid. M.Sc. thesis (in Hebrew), Sackler School of Medicine, Tel-Aviv University.
Gribnau J, Hochedlinger K, Hata K, Li E, Jaenisch R. (2003) Asynchronous replication timing of imprinted loci is independent of DNA methylation, but consistent with differential subnuclear localization. Genes Dev 17:759-773.
Jones P, Baylin S B. (2002) The fundamental role of epigenetic events in cancer. Nature Rev Genet 3:415-428.
Kane M F, Loda M, Gaida G M, Lipman J, Mishra R, Goldman H, Jessup J M, Kolodner R. (1997) Methylation of the hMLH1 promoter correlates with lack of expression of hMLH1 in sporadic colon tumors and mismatch repair-defective human tumor cell lines. Cancer Res 57:808-811.
Korenstein-Ilan A, Amiel A, Lalezari S, Lishner M, Avivi L. (2002) Allele specific replication associated with aneuploidy in blood cells of patients with hematological malignancies. Cancer Genet Cytogenet 139:97-103.
Litmanovitch T, Altaras M M, Dotan A, Avivi L. (1998) Asynchronous replication of homologous a-satellite DNA loci in man is associated with non-disjunction. Cytogenet Cell Genet 81:26-35.
Mukherjee A B, Thomas S (1997) A longitudinal study of human age-related chromosomal analysis in skin fibroblasts. Exp Cell Res 235:161-169.
Ofir R, Wong A C C, Mcdermid H E, Scorecki K L, Selig S (1999) Position effect of human telomeric repeats on replication timing. Proc Natl Acad Sci USA 96:11434-11439.
Randhawa G S, Cui H, Barletta J A, Strichman-Almashanu L Z., Talpaz M, Kantarjian H, Deisseroth A B, Champlin R C, Feinberg A P. (1998) Loss of imprinting in disease progression in chronic myelogenous leukemia. Blood 91:3144-3147.
Reish O, Gal R, Gaber E, Sher C, Bistritzer T, Amiel A (2002) Asynchronous replication of biallelically expressed loci: A new phenomenon in Turner syndrome. Genet Med 4:439-443.
Selig S, Okumura K, Ward D C, Cedar H. (1992) Delination of DNA replication time zones by fluorescence in situ hybridization. EMBO J 11:1217-1225.
Senn M (2003) Altered mode of allelic replication and increased aneuploidy levels in peripheral blood lymphocytes of Down-syndrome subjects. M.Sc. thesis (in Hebrew), Sackler School of Medicine, Tel-Aviv University.
Singh N, Ebrahimi F A, Gimelbrant A A, Ensminger A W, Tackett M R, Qi P, Gribnau J, Chess A. (2003) Coordination of the random asynchronous replication autosomal loci. Nat Genet 33:359-341.

1. A method of diagnosing cancer, the method comprising, determining in at least one locus of a cell of an individual in need thereof an interallelic epigenetic pattern, wherein an alteration in said interallelic epigenetic pattern compared to said interallelic epigenetic pattern of said at least one locus in a cell of an unaffected individual is indicative of the cancer, thereby diagnosing the cancer.

2. A method of prenatally identifying a chromosomal imbalance in a conceptus, the method comprising, determining in at least one locus of a maternal cell of a pregnant female carrying the conceptus an interallelic epigenetic pattern, wherein an alteration in said interallelic epigenetic pattern compared to said interallelic epigenetic pattern of said at least one locus in a cell of an individual devoid of the chromosomal imbalance is indicative the chromosomal imbalance in the conceptus.

3. A method of prenatally identifying a chromosomal imbalance in a conceptus with the proviso that the chromosomal imbalance is not an imprinting-associated chromosomal imbalance, the method comprising, determining in at least one locus of a cell of the conceptus or a maternal cell of a pregnant female carrying the conceptus an interallelic epigenetic pattern, wherein an alteration in said interallelic epigenetic pattern compared to said interallelic epigenetic pattern of said at least one locus in a cell of an individual devoid of the chromosomal imbalance is indicative of the chromosomal imbalance in the conceptus.

4. A method of prenatally identifying an imprinting-associated chromosomal imbalance in a conceptus, the method comprising determining in at least one locus of a cell of the conceptus or a maternal cell of a pregnant female carrying the conceptus an interallelic epigenetic pattern with the proviso that said at least one locus is a monoallelically expressed locus, wherein an alteration in said interallelic epigenetic pattern compared to said interallelic epigenetic pattern of said at least one locus in a cell of an individual devoid of the chromosomal imbalance is indicative of the imprinting-associated chromosomal imbalance in the conceptus.

5. A method of identifying a chromosomal imbalance mosaicism in an individual in need thereof, the method comprising determining in at least one locus of a cell of the individual an interallelic epigenetic pattern, wherein an alteration in said interallelic epigenetic pattern compared to said interallelic epigenetic pattern of said at least one locus in a cell of an individual devoid of the chromosomal imbalance mosaicism is indicative the chromosomal imbalance mosaicism in the individual.

6. A method of prenatally identifying a chromosomal imbalance of a conceptus with the proviso that the chromosomal imbalance is not an imprinting-associated chromosomal imbalance, the method comprising, determining in at least one locus of a cell of the conceptus an interallelic replication pattern, wherein an alteration in said interallelic replication pattern compared to said interallelic replication pattern of said at least one locus in a cell of an individual devoid of the chromosomal imbalance is indicative of the chromosomal imbalance in the conceptus.

7. A method of prenatally identifying a chromosomal imbalance of a conceptus, the method comprising, determining in at least one locus of a maternal cell of a pregnant female carrying the conceptus an interallelic replication pattern, wherein an alteration in said interallelic replication pattern compared to said interallelic replication pattern of said at least one locus in a cell of an individual devoid of the chromosomal imbalance is indicative of the chromosomal imbalance in the conceptus.

8. The method of claims 1, wherein said epigenetic pattern is selected from the group consisting of DNA methylation, chromatic methylation and chromatin acetylation.

9-18. (canceled)

19. The method of claim 3, wherein said at least one locus is selected from the group consisting of a biallelically expressed locus, a monoallelically expressed locus and a non-coding locus.

20. The method of claim 4, wherein said at least one locus is a biallelically expressed locus and/or a non-coding locus.

21. The method of claim 19, wherein said biallelically expressed locus comprises a tumor-suppressor gene, an oncogene, a transcription factor and a housekeeping gene.

22. The method of claim 19, wherein said biallelically expressed locus comprises a gene selected from the group consisting of HER2, CMYC, TP53, RB1, TP53, AML1, STS and KAL1.

23. The method of claim 19, wherein said monoallelically expressed locus comprises an imprinted gene, a gene subjected to chromosome X inactivation and/or random monoallelically expressed autosomal gene.

24-26. (canceled)

27. The method of claim 19, wherein said monoallelically expressed locus is selected from the group consisting of 15q11-13 and 11p15.

28. The method of claim 19, wherein said non-coding locus comprises a nucleic acid sequence associated with chromosome segregation.

29-36. (canceled)

37. The method of claim 3, wherein said cell of the conceptus is derived from amniotic fluid, CVS, cord blood and placenta.

38. The method of claim 3, wherein said maternal cell is a blood cell.

39-40. (canceled)

41. The method of claim 3, wherein said chromosomal imbalance is gain of a whole chromosome or a portion thereof, loss of a whole chromosome or a portion thereof, duplication, deletion, microdeletion and imbalanced rearrangement.

42. (canceled)

43. The method of claim 4, wherein said imprinting-associated chromosomal imbalance is Prader-Willi syndrome (PWS), Angelman syndrome (AS) and Beckwith-Wiedemann syndrome (BWS).

44. The method of claim 3, wherein said chromosomal imbalance is Down syndrome, Turner syndrome, Edwards' syndrome, Patau's syndrome, Di-George syndrome, Williams syndrome (WS) and/or Duchenne muscular dystrophy.

45-48. (canceled)

49. The method of claim 4, wherein said epigenetic pattern is selected from the group consisting of DNA methylation, chromatic methylation and chromatin acetylation.

50. The method of claim 20, wherein said biallelically expressed locus comprises a tumor-suppressor gene, an oncogene, a transcription factor and a housekeeping gene.

51. The method of claim 20, wherein said biallelically expressed locus comprises a gene selected from the group consisting of HER2, CMYC, TP53, RB1, TP53, AML1, STS and KAL1.
